(19) United States
(12) Patent Application Publication
(10) Pub. No.: US 2020/0276102 A1
(43) Pub. Date: Sep. 3, 2020

(54) SPECIFIC 7-AMINO-1,2,3,4-TETRAHYDROQUINOLINES, METHOD, AND COMPOSITION

(71) Applicant: L'Oreal, Paris (FR)

(72) Inventors: Aziz Fadli, Aulnay-sous-Bois (FR); Zhibo Liu, Aulnay-sous-Bois (FR); Céline Richardson, Aulnay-sous-Bois (FR)

(73) Assignee: L'Oreal, Paris (FR)

(21) Appl. No.: 16/755,832

(22) PCT Filed: Oct. 5, 2018
(86) PCT No.: PCT/EP2018/077088

§ 371 Date: Apr. 13, 2020

(30) Foreign Application Priority Data
Oct. 13, 2017 (FR) 1759604

(51) Int. Cl. A61K 8/49; A61Q 5/10

(52) U.S. Cl. CPC A61K 8/4926; A61Q 5/10; A61K 8/4946; A61K 2800/10; A61K 2800/882; A61K 2800/4324; A61K 2800/4322; A61K 8/4933

(57) ABSTRACT

The present patent application relates to a process for dyeing keratin fibers, in particular human keratin fibers such as the hair, comprising a step of applying to said keratin fibers one or more 7-amino-1,2,3,4-tetrahydroquinoline derivatives substituted in position 8, to dye compositions comprising such 7-amino-1,2,3,4-tetrahydroquinolines, and also to the devices using these compounds.

The present patent application relates to a process for dyeing keratin fibers, in particular human keratin fibers such as the hair, comprising a step of applying to said keratin fibers one or more 7-amino-1,2,3,4-tetrahydroquinoline derivatives substituted in position 8, to dye compositions comprising such 7-amino-1,2,3,4-tetrahydroquinolines, and also to the devices using these compounds.

It is known practice to dye keratin fibers, and in particular human hair, with dye compositions containing oxidation dye precursors, which are generally known as oxidation bases, such as ortho- or para-phenylenediamines, ortho- or para-aminophenols and heterocyclic compounds. These oxidation bases are colorless or weakly colored compounds which, when combined with oxidizing products, are able to produce colored compounds by a process of oxidative condensation.

It is also known that the shades obtained with these oxidation bases may be varied by combining them with couplers or color modifiers, the latter being notably chosen from aromatic meta-diaminobenzenes, meta-aminophenols, meta-diphenols and certain heterocyclic compounds such as indole compounds.

The variety of molecules used as oxidation bases and couplers allows a wide range of colors to be obtained.

The “permanent” coloring obtained by means of these oxidation dyes should moreover satisfy a certain number of requirements. Thus, it should have no toxicological drawbacks, it should allow shades to be obtained in the desired intensity, and it should show good resistance to external agents such as light, bad weather, washing, temporary or permanent hair shaping treatments, perspiration and rubbing.

The dyes should also allow gray hair to be covered and, finally, they should be as unselective as possible, i.e. they should make it possible to produce the smallest possible coloring differences along the same lock of keratin fiber, which in general is differently sensitized (i.e. damaged) between its end and its root.

Heterocyclic oxidation bases make it possible to obtain a wide range of colors, but their combinations with conventional couplers are occasionally lacking in homogeneity and chromaticity and the selectivities are often high.

Certain 7-amino-1,2,3,4-tetrahydroquinoline derivatives are known as dyes for polyesters (DE 2941512). Other derivatives have been used for their therapeutic application (see, for example: vanilloid receptor modulator: WO 2003/068749; 5HT1A, 5HT1B and HTip receptor antagonists: WO 98/47868; capsaicin receptor modulator: WO 2005/023807; NO inhibitor: US 2008/0234237, and CCR5 receptor agonist or antagonist: WO 00/06146).

It is known practice in hair dyeing to use 7-amino-1,2,3,4-tetrahydroquinoline derivatives as couplers (WO 2008/025240). However, the colorings obtained with these couplers are not always satisfactory. Specifically, whether in terms of solubility, color build-up, chromaticity, fastness, persistence (with respect to washing, bad weather or light) and/or selectivity of the color (root/end “homogeneity” of the color), these couplers do not always afford the user satisfaction.

There is thus a real need for oxidation couplers that can dye keratin fibers in an intense, fast, sparingly selective and chromatic manner, with good build-up of the color, and which are capable of giving colorings that are resistant to the various attacking factors to which the fibers may be subjected, such as bad weather, washing and perspiration.

These aims are achieved with the present invention, one subject of which is notably a process for dyeing keratin fibers, in particular human keratin fibers such as the hair, comprising at least one step i) of applying to said keratin fibers one or more compounds of formula (I) below, and also the organic or mineral acid or base salts thereof, the tautomeric forms thereof, the optical isomers thereof, the geometrical isomers thereof and/or the solvates thereof such as the hydrates thereof:

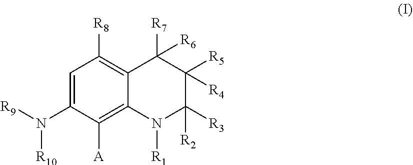

in which formula (I):
A represents:
i) a halogen atom,
ii) a carboxyl radical —COOH or carboxylate radical—COO⁻M⁺,
iii) a sulfonic radical —SO₃H or sulfonate radical—SO₃⁻M⁺, or alkylsulfonyl radical —S(O)₂—R with R representing a linear or branched (C₁-C₄)alkyl group,
iv) a group —C(R₁₂)(R₁₃)—R₁₁ or —C(R₁₂)═C(R₁₁)—R₁₃, in which R₁₁, R₁₂ and R₁₃, which may be identical or different, represent:
   a hydrogen atom,
   a halogen atom such as fluorine, chlorine, bromine or iodine, preferably fluorine,
   a hydroxyl radical,
   a linear or branched C₁-C₁₄ alkyl or C₂-C₁₄ alkenyl, in particular C₁-C₈ alkyl or C₂-C₈ alkenyl, preferably C₁-C₆ alkyl or C₂-C₆ alkenyl group, said alkyl or alkenyl group:
      optionally interrupted with one or more heteroatoms or groups, which may be identical or different, chosen from —O—, —S—, —S(O)—, —S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N═, ═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—N(H)—, and/or said alkyl or alkenyl group being
      optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) halo such as fluoro —F, vi) sulfonic —SO₃H, or sulfonate—SO₃⁻M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl; ix) an ammonium radical—N+RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
   or else R₁₁ forms with R₉, the nitrogen atom which bears R₉ and the carbon atoms which bear —N(R₉)—R₁₀ and R₁₁, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical—N+RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O—),
v) a group —W—R₁₄ in which:
   W represents an oxygen or sulfur atom or a divalent group chosen from: —N(R′₁₄)—, —S(O)— and —S(O)₂—,
   R₁₄ and R′₁₄ independently represent:
      a hydrogen atom,
      a linear or branched C₁-C₁₄ alkyl or C₂-C₁₄ alkenyl group, in particular C₁-C₈, preferably C₁-C₆, said alkyl or alkenyl group being:
         optionally interrupted with one or more heteroatoms or groups, which may be identical or different, chosen from —O—, —S—, —S(O)—, —S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N═, ═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—N(H)—,
         and/or said alkyl or alkenyl group being optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) halo such as fluoro —F, vi) sulfonic —SO₃H, or sulfonate —SO₃⁻M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl; ix) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
   or else R₁₄ forms with R₉, the nitrogen atom which bears R₉ and the carbon atoms which bear —N(R₉)(R₁₀) and —W—, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O—),
vi) a radical —C(X′)—R₁₁, preferably carbonyl —C(O)—R₁₁, with R₁₁ as defined previously,
vii) a radical —C(X′)—X″—R₁₁, preferably ester —C(O)—O—R₁₁, thioester —C(O)—S—R₁₁, dithioester —C(S)—S—R₁₁, with R₁₁ as defined previously, —X″—C(X′)—R₁₁, preferably amide —N(R′)—C(O)—R₁₁ or —X″—C(X′)—X—R₁₁, preferably carbamate —N(R′)—C(O)—O—R₁₁,
   with X as defined previously, and X′ and X″, which may be identical or different, represent an oxygen or sulfur atom or a group N(R′) with R′ representing a hydrogen atom or a C₁-C₄ alkyl group; preferably, X′ represents an oxygen atom and X″ represents an oxygen or sulfur atom or a group N(R′) with R′ representing a hydrogen atom or a C₁-C₄ alkyl group;
viii) an optionally substituted cationic or noncationic (hetero)cycle,
ix) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl,
R₁ represents:
   a hydrogen atom,
   a saturated or unsaturated, linear or branched C₁-C₆ alkyl radical, said alkyl radical:
      optionally comprising one or more heteroatoms or groups, which may be identical or different, chosen from —O—, —S—, —S(O)—, —S(O)₂—, —N(H)—, —N(R)—, ═N—R—, —R—N═ and —C(X)— with R as defined previously, and with X representing an oxygen or sulfur atom or N—R, or combinations of said heteroatoms or groups, preferably chosen from: —O—C(O)—, —C(O)—O—, —C(O)—N(H)—, —N(H)—C(O)—, —C(O)—N═, ═N—C(O)—, —N(H)—C(O)—N(H)—, —O—C(O)—N(H)—, —N(H)—C(O)—O—, —N(H)—C(NH)—NH—, and/or said alkyl radical being
      optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) cationic or noncationic (hetero)cycle;
preferably, R₁ represents a hydrogen atom;
R₂, R₃, R₄, R₅, R₆ and R₇, which may be identical or different, represent:
   a hydrogen atom,
   a hydroxyl radical,
   a saturated or unsaturated, linear or branched C₁-C₆ alkyl radical, said alkyl radical:
      optionally comprising one or more heteroatoms or groups, which may be identical or different, chosen from —O—, —S—, —S(O)—, —S(O)₂—, —N(H)—, —N(R)—, ═N—R—, —R—N═ and —C(X)— with R as defined previously, and with X representing an oxygen or sulfur atom or N—R, or combinations of said heteroatoms or groups, preferably chosen from: —O—C(O)—, —C(O)—O—, —C(O)—N(H)—, —N(H)—C(O)—, —C(O)—N═, ═N—C(O)—, —N(H)—C(O)—N(H)—, —O—C(O)—N(H)—, —N(H)—C(O)—O—, —N(H)—C(NH)—NH—, and/or said alkyl radical being
      optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino,
   an aromatic or nonaromatic, saturated or unsaturated, 5- to 8-membered (hetero)cycle, optionally substituted with one or more radicals, which may be identical or different, chosen from the following radicals: i) hydroxyl, ii) C₁-C₆ (hydroxy)alkyl, iii) C₁-C₆ (hydroxy)alkoxy, iii) amino —NH₂, iv) C₁-C₆ mono and/or di(hydroxy)alkylamino, and at least one of the members of which may represent a divalent oxo radical;
      preferably, R₂ to R₇ represent a hydrogen atom;
R₈ represents:
   a hydrogen atom,
   a halogen atom such as fluorine, chlorine, bromine or iodine, preferably chlorine, fluorine or bromine,
   a hydroxyl radical,
   a (C₁-C₈)alkylcarbonyl radical,
   a saturated or unsaturated, linear or branched C₁-C₈ alkyl radical, said alkyl radical:
      optionally comprising one or more heteroatoms or groups, which may be identical or different, chosen from —O—, —S—, —S(O)—, —S(O)₂—, —N(H)—, —N(R)—, ═N—R—, —R—N═ and —C(X)— with R as defined previously, and with X representing an oxygen or sulfur atom or N—R, or combinations of said heteroatoms or groups, preferably chosen from: —O—C(O)—, —C(O)—O—, —C(O)—N(H)—, —N(H)—C(O)—, —C(O)—N═, ═N—C(O)—, —N(H)—C(O)—N(H)—, —O—C(O)—N(H)—, —N(H)—C(O)—O—, —N(H)—C(NH)—NH—, and/or said alkyl radical being
      optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) cationic or noncationic (hetero)cycle;
preferentially, R₈ represents a hydrogen atom;
R₉ and R₁₀, which may be identical or different, represent:
   a hydrogen atom,
   a saturated or unsaturated, linear or branched C₁-C₆ alkyl radical, said alkyl radical:
      optionally comprising one or more heteroatoms or groups, which may be identical or different, chosen from —O—, —S—, —S(O)—, —S(O)₂—, —N(H)—, —N(R)—, ═N—R—, —R—N═ and —C(X)— with R as defined previously, and with X representing an oxygen or sulfur atom or N—R, or combinations of said heteroatoms or groups, preferably chosen from: —O—C(O)—, —C(O)—O—, —C(O)—N(H)—, —N(H)—C(O)—, —C(O)—N═, ═N—C(O)—, —N(H)—C(O)—N(H)—, —O—C(O)—N(H)—, —N(H)—C(O)—O—, —N(H)—C(NH)—NH—, and/or said alkyl radical being
      optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) cationic or noncationic (hetero)cycle;
   or R₉ and R₁₀ form, with the nitrogen atom that bears them, a 5- to 8-membered heterocycle, optionally substituted with one or more radicals, which may be identical or different, chosen from the following radicals: i) hydroxyl, ii) C₁-C₁₀ (hydroxy)alkyl, iii) C₁-C₆ (hydroxy)alkoxy, iv) amino —NH₂, v) C₁-C₄ mono and/or di(hydroxy)alkylamino, vi) thiol —SH, vii) C₁-C₄ alkylthiol, viii) carboxyl —COOH or carboxylate —COO⁻, M⁺, ix) C₁-C₄ alkylcarbonyl, x) sulfonic —SO₃H or sulfonate —SO₃⁻, M⁺, xi) amido —NH—C(O)CH₃, and at least one of the members of said heterocycle possibly representing a divalent oxo radical;
      preferably, R₉ and R₁₀ represent a hydrogen atom;
An⁻ denotes an anion or a mixture of anions for ensuring the electrical neutrality of the molecule, preferably chosen from halide ions such as bromide or chloride, methyl sulfate or toluenesulfonate ions, or a mixture of these ions;
M⁺ represents a cation or a mixture of cations for ensuring the electrical neutrality of the molecule, preferably chosen from sodium, potassium, calcium and ammonium.

Preferably, R₁, R₂, R₃, R₄, R₅, R₆ and R₇ represent a hydrogen atom.

When the compounds of formula (I) include both cationic and anionic radicals, An⁻ or M⁺ are necessary only when the charges on the anionic groups and on the cationic groups do not neutralize each other.

The compounds of formula (I) according to the invention lead to a wide range of colors in oxidation dyeing. These couplers notably make it possible to enlarge the color range. Furthermore, these 7-amino-1,2,3,4-tetrahydroquinoline derivatives substituted in position 8 make it possible to obtain colorings with varied shades, notably natural light shades and natural dark shades. These heterocyclic couplers furthermore show good solubility.

The compounds according to the invention can thus give colorings that are resistant to the various attacking factors to which keratin fibers may be subjected, such as bad weather, light, washing and/or perspiration.

Furthermore, the oxidation couplers of formula (I) according to the invention make it possible to dye keratin fibers satisfactorily, notably leading to powerful, fast, chromatic and sparingly selective colorings, and/or colorings with a good color build-up.

A subject of the invention is also the use of the compounds of formula (I), and also the organic or mineral acid or base salts thereof, the tautomeric forms thereof, the optical isomers thereof, the geometrical isomers thereof and/or the solvates thereof, as defined previously, for dyeing keratin fibers, in particular human keratin fibers such as the hair.

A subject of the invention is also a kit or multi-compartment device comprising at least one compound of formula (I) as defined previously.

Other features, aspects, subjects and advantages of the present invention will emerge even more clearly on reading the description and the examples that follow.

In the text hereinbelow, and unless otherwise indicated:
the limits of a range of values are included in that range, notably in the expressions “between . . . and . . . ” and “ranging from . . . to . . . ”;
the expressions “at least one” and “at least” used in the present description are equivalent, respectively, to the expressions “one or more” and “greater than or equal to”;
according to the present patent application, the term “keratin fibers” mainly denotes human keratin fibers and more particularly the hair;
the term “anion” means a cosmetically acceptable anion or anionic group derived from an organic or mineral acid salt associated with the cationic charge of the compound of formula (I); more particularly, the anion is chosen from: i) halides such as chloride or bromide; ii) alkylsulfonates, including C₁-C₆ alkylsulfonates: Alk-S(O)₂O⁻ such as methylsulfonate or mesylate and ethylsulfonate; iii) arylsulfonates: Ar—S(O)₂O⁻ such as benzenesulfonate and toluenesulfonate or tosylate; iv) citrate; v) succinate; vi) tartrate; vii) lactate; viii) alkyl sulfates: Alk-O—S(O)O⁻ such as methyl sulfate and ethyl sulfate; ix) aryl sulfates: Ar—O—S(O)O⁻ such as benzene sulfate and toluene sulfate; x) alkoxy sulfates: Alk-O—S(O)₂O⁻ such as methoxy sulfate and ethoxy sulfate; xi) aryloxy sulfates: Ar—O—S(O)₂O⁻, xii) phosphates O═P(OH)₂—O⁻, O═P(O⁻)₂—OH O═P(O⁻)₃, HO—[P(O)(O⁻)]_{w}—P(O)(O⁻)₂ with w being an integer; xiii) acetate; xiv) triflate; xv) borates such as tetrafluoroborate; xvi) disulfate S(O)₂O₂⁻; xvii) carbonate; and xviii) hydrogen carbonate;
more particularly, the cosmetically acceptable anions are chosen in particular from halides such as chloride; methosulfates; alkylsulfonates: Alk-S(O)₂O⁻ such as methylsulfonate or mesylate and ethylsulfonate; arylsulfonates: Ar—S(O)₂O⁻ such as benzenesulfonate and toluenesulfonate or tosylate; citrate; succinate; tartrate; lactate; alkyl sulfates: Alk-O—S(O)O⁻ such as methyl sulfate; aryl sulfates such as benzene sulfate and toluene sulfate; phosphate; acetate; triflate; borates such as tetrafluoroborate; carbonate; and hydrogen carbonate;
As the anion, derived from the organic or mineral acid salt, ensures the electrical neutrality of the molecule, it is understood that when the anion comprises several anionic charges, then the same anion may serve for the electrical neutrality of several cationic groups in the same molecule or else may serve for the electrical neutrality of several molecules; for example, a compound of formula (I) which possibly contains two cationic groups may contain either two “singly charged” anionic counterions or a “doubly charged” anionic counterion such as S(O)₂O₂⁻ or O═P(O⁻)₂—OH;
The term “cation” means a cosmetically acceptable cation or cationic group derived from an organic or mineral base salt associated with the anionic charge of the compound of formula (I); more particularly, the cation is chosen from i) alkali metals such as Na⁺ and K⁺, ii) alkaline-earth metals such as Ca⁺⁺ and Mg^{++,} and iii) ammoniums such as RₐR_{b}R_{c}R_{d}N⁺ with Rₐ, R_{b}, R_{c} and R_{d}, which may be identical or different, representing a hydrogen atom or a hydroxyl or (C₁-C₈)alkyl group.
the term “alkyl” means a linear, branched or cyclic, saturated hydrocarbon-based radical, preferably of C₁-C₈, more preferentially C₁-C₆ and even more preferentially C₁-C₄, such as methyl, ethyl, propyl, isobutyl, isopropyl or tert-butyl;
the term “alkenyl” means a linear or branched, preferably C₁-C₈ and more preferentially C₁-C₆ hydrocarbon-based radical, comprising at least one carbon-carbon double bond or at least one carbon-carbon triple bond, preferably comprising at least one carbon-carbon double bond and even more preferentially comprising a carbon-carbon double bond;
the term “hydroxyalkyl” means an alkyl group as defined previously, substituted with one or more hydroxyl groups, preferably a (C₁-C₈)alkyl and more preferentially (C₁-C₆)alkyl group substituted with a hydroxyl group, such as hydroxyethyl;
the term “(hydroxy)alkyl” means an alkyl or hydroxyalkyl group as defined previously;
the term “alkoxy” means a group —O-alkyl with alkyl as defined previously; in particular, “alkoxy” denotes a methoxy or ethoxy group;
the term “hydroxyalkoxy” means an alkoxy group as defined previously, substituted with one or more hydroxyl groups, preferably a (C₁-C₆)alkoxy group substituted with a hydroxyl group, such as β-hydroxyethoxy;
the term “(hydroxy)alkoxy” means an alkoxy or hydroxyalkoxy group as defined previously; the term “mono(hydroxy)alkylamino” means an —NH-(hydroxy)alkyl group with (hydroxy)alkyl as defined previously; in particular, “mono(hydroxy)alkylamino” denotes a methylamino, ethylamino, 2-hydroxyethylamino or hydroxymethylamino group;
the term “di(hydroxy)alkylamino” means an —N-((hydroxy)alkyl)(hydroxy)alkyl group with each (hydroxy)alkyl being as defined previously; in particular, “dialkylamino” denotes a dimethylamino, diethylamino or bis(2-hydroxyethylamino) group;
the term “heterocycle” or “heterocyclic” radical means a noncationic cyclic radical which is preferably from 5- to 14-membered, preferably comprising from 1 to 5 heteroatoms such as O, S or N, said radical possibly being saturated, unsaturated or aromatic and at least one of the members possibly denoting an oxo radical; more preferentially, said heterocyclic radical is monocyclic or bicyclic, from 5- to 10-membered and comprising from 1 to 3 heteroatoms chosen from N, O and S, more particularly N and O, for instance imidazolyls, pyridinyls, piperazinyls, pyrrolidinyls, morpholinyles, pyrimidinyls, thiazolyls, benzimidazolyls, benzothiazolyls, oxazolyls, benzotriazolyls, pyrazolyls, triazolyls, benzoxazolyls, piperidinyls, pyrrolyls, and oxazolidinylones such as 1,3-oxazolidin-2-one;
the term “optionally substituted heterocycle” or “optionally substituted heterocyclic” radical means a “heterocycle” or “heterocyclic” radical as defined previously, said radical being optionally substituted with one or more radicals, which may be identical or different, chosen from the following radicals: i) hydroxyl, ii) C₁-C₁₀ (hydroxy)alkyl, iii) C₁-C₆ (hydroxy)alkoxy, iv) amino —NH₂, v) C₁-C₄ mono and/or di(hydroxy)alkylamino, vi) thiol —SH, vii) C₁-C₄ alkylthiol, viii) carboxyl —COOH or carboxylate —COO⁻, M⁺ ix) C₁-C₄ alkylcarbonyl, x) C₁-C₆ alkoxycarbonyl, xi) sulfonic —SO₃H or sulfonate —SO₃⁻, M⁺, xi) amido —NH—C(O)R with R denoting a C₁-C₄ alkyl radical such as methyl, xii) halogen such as fluorine, chlorine, bromine or iodine;
the term “(hetero)cycle” or “(hetero)cyclic” radical means a saturated, unsaturated or aromatic hydrocarbon-based cyclic radical which is preferably from 5- to 14-membered, or a “heterocycle” radical as defined previously; more preferentially, said hydrocarbon-based cyclic radical is monocyclic or bicyclic, and from 5- to 10-membered, for instance a phenyl radical;
the term “cationic heterocycle” or “cationic heterocyclic” radical means a “heterocycle” or “heterocyclic” radical as defined previously and including a cationic charge, said cationic charge possibly constituting one of the members of the cyclic radical (endocyclic cationic charge) or may be a substituent on said ring (exocyclic cationic charge); the endocyclic cationic charge denotes a divalent radical —N⁺(Rₐ)—, An⁻ or —N⁺(Rₐ)(R_{b})—, An⁻ depending on whether the ring is saturated, unsaturated or aromatic or already engaged in a covalent bond via the cationic nitrogen atom, Rₐ and R_{b} independently denoting a C₁-C₆ (hydroxy)alkyl and preferably C₁-C₄ alkyl radical such as methyl; the exocyclic cationic charge denotes a trialkylammonium radical such as a radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
the term “optionally substituted cationic heterocycle” or “optionally substituted cationic heterocyclic” radical means an “optionally substituted heterocycle” or “optionally substituted heterocyclic” radical as defined previously and including a cationic charge as defined previously.

### Dyeing Process, Compounds of Formula (I)

The present invention relates to a process for dyeing keratin fibers, in particular human keratin fibers such as the hair, comprising at least one step i) of applying to said keratin fibers one or more compounds of formula (I) as described previously, and also the organic or mineral acid or base salts thereof, the tautomeric forms thereof, the optical isomers thereof, the geometrical isomers thereof and/or the solvates thereof such as the hydrates thereof.

The compounds of formula (I) according to the invention are mandatorily substituted at C-8. In other words, the group A cannot represent a hydrogen atom.

The compounds of formula (I) may be in the form of an organic or mineral acid or base salt.

The term “organic or mineral acid salt” more particularly means salts chosen from addition salts with a cosmetically acceptable acid such as salts derived from i) hydrochloric acid HCl, ii) hydrobromic acid HBr, iii) sulfuric acid H₂SO₄, iv) alkylsulfonic acids: Alk-S(O)₂OH such as methylsulfonic acid and ethylsulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)—OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; xv) tetrafluoroboric acid HBF₄; and xvi) hydriodic acid HI.

More particularly, the compounds of formula (I) are optionally salified with strong mineral acids, such as HCl, HBr, HI, H₂SO₄ or H₃PO₄, or organic acids, for instance acetic acid, lactic acid, tartaric acid, citric acid, succinic acid, benzenesulfonic acid, para-toluenesulfonic acid, formic acid or methanesulfonic acid.

The term “organic or mineral base salt” more particularly means salts chosen from addition salts with a cosmetically acceptable base, for instance alkali metal hydroxides such as sodium hydroxide or potassium hydroxide, ammonia, amines or alkanolamines, or with a cosmetically acceptable anion as defined previously.

The compounds of formula (I) may also be in the form of solvates, for example a hydrate or a solvate of a linear or branched alcohol such as ethanol or isopropanol.

Preferably, the ammonium radical —N⁺RR′R″, An⁻, with R, R′ and R″, which may be identical or different, representing a (C₁-C₄)alkyl group optionally substituted with one or more hydroxyl groups, according to the definition of group A of the compound of formula (I), is chosen from trimethylammonium, triethylammonium, dimethylethylammonium, diethylmethylammonium, diisopropylmethylammonium, diethylpropylammonium, hydroxyethyldiethylammonium, β,β-dihydroxyethylmethylammonium and β,β,β-trihydroxyethylammonium radicals; preferably from trimethylammonium, triethylammonium, dimethylethylammonium, diethylmethylammonium, diisopropylmethylammonium and hydroxyethyldiethylammonium radicals and even more preferentially denotes a trimethylammonium radical.

Preferably, the cationic heterocycle according to the definition of group A of the compound of formula (I) is chosen from imidazoliums, pyridiniums, piperaziniums, pyrrolidiniums, morpholiniums, pyrimidiniums, thiazoliums, benzimidazoliums, benzothiazoliums, oxazoliums, benzotriazoliums, pyrazoliums, triazoliums and benzoxazoliums; preferably from imidazoliums, pyridiniums, piperaziniums, pyrrolidiniums, morpholiniums, pyrimidiniums, thiazoliums and benzimidazoliums, and even more preferentially denotes an imidazolium or a piperazinium.

Preferably, the noncationic heterocycle according to the definition of group A of the compound of formula (I) is chosen from imidazoles, pyridines, piperazines, pyrrolidines, morpholines, pyrimidines, thiazoles, benzimidazoles, benzothiazoles, oxazoles, benzotriazoles, pyrazoles, triazoles, benzoxazoles, piperidines, pyrroles and oxazolidinones; preferably from morpholines, piperidines, pyrrolidines, imidazoles, pyrroles, piperazines and 1,3-oxazolidin-2-ones.

Preferably, R₁, R₂, R₃, R₄, R₅, R₆, R₇ and R₈, which may be identical or different, represent:
a hydrogen atom,
a saturated or unsaturated, linear or branched C₁-C₆ alkyl radical, said alkyl radical being optionally substituted with one or more radicals, which may be identical or different, chosen from the following radicals: i) hydroxyl, ii) C₁-C₄ alkoxy, iii) amino —NH₂, iv) C₁-C₆ mono and/or di(hydroxy)alkylamino.

More preferentially, R₁, R₂, R₃, R₄, R₅, R₆, R₇ and R₈ represent a hydrogen atom.

Preferably, R₉ and R₁₀, which may be identical or different, represent:
a hydrogen atom,
a saturated or unsaturated, linear or branched C₁-C₆ alkyl radical, said alkyl radical being optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino.

More preferentially, R₉ and R₁₀ are identical and represent a hydrogen atom.

According to a particular embodiment of the invention, the compounds of formula (I) are such that A represents a group —C(R₁₂)(R₁₃)—R₁₁ and R₁₁ forms with R₉, the nitrogen atom which bears R₉ and the carbon atoms which bear —N(R₉)—R₁₀ and R₁₁, a 5- to 8-membered cationic or noncationic heterocycle, said heterocycle being:
optionally substituted with one or more radicals, which may be identical or different, chosen from the following radicals: i) hydroxyl, ii) C₁-C₄ alkyl, iii) C₁-C₄ alkoxy and iv) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
optionally at least one of the members of said heterocycle possibly representing a divalent oxo radical —C(O)—;
   R₁₂ and R₁₃ being as defined previously and preferably denoting a hydrogen atom.

According to this embodiment, A represents a group —CH₂—R₁₁ and R₁₁ forms with R₉, the nitrogen atom which bears R₉ and the carbon atoms which bear —N(R₉)—R₁₀ and R₁₁, a 5- or 6-membered, preferably 6-membered, noncationic heterocycle such as a piperidine ring, said heterocycle being optionally substituted with one or more radicals, which may be identical or different, chosen from the following radicals: i) hydroxyl, ii) C₁-C₄ alkyl, iii) C₁-C₄ alkoxy.

According to another embodiment of the invention, the compounds of formula (I) are chosen from the compounds of formula (IA) below, and also the geometrical or optical isomers thereof, the tautomers thereof, the organic or mineral acid or base salts thereof or the solvates thereof such as hydrates:

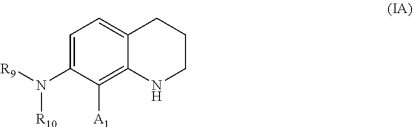

in which A₁ represents:
a) a radical —(Y)ₚ—B with Y denoting an oxygen or sulfur atom or —S(O)₂—, p denoting an integer equal to 0 or 1, and B denoting either a C₁-C₆ alkyl or alkenyl radical, said alkyl or alkenyl radical being optionally substituted with one or more radicals chosen independently from: i) halogen such as fluorine, ii) hydroxyl, iii) SO₃H or SO₃⁻, M⁺ with M⁺ as defined previously, iv) ammonium —N⁺RR′R″, An⁻, with R, R′ and R″, which may be identical or different, representing a (C₁-C₄)alkyl group such as methyl, and in particular trimethylammonium, or B denotes a 5- or 6-membered cationic heterocycle such as imidazolium or piperazinium;
b) a radical chosen from i) SO₃H or SO₃⁻, M⁺ with M⁺ as defined previously, ii) carboxyl CO₂H or CO₂⁻, M⁺ with M⁺ as defined previously, iii) alkoxycarbonyl —CO₂R_{f} in which R_{f} denotes a C₁-C₄ alkyl radical such as methyl, iv) alkylcarbonyl —C(O)—R_{f} with R_{f} as defined above;
c) a radical —N(R′₁₄)—R₁₄ with R′₁₄ and R₁₄ as defined previously and preferably with R′₁₄ denoting a hydrogen atom or a C₁-C₆ (hydroxy)alkyl radical such as methyl or 2-hydroxyethyl and R₁₄ denoting a radical chosen from i) alkylcarbonyl —C(O)—R_{f} with R_{f} as defined above, ii) alkoxycarbonyl —C(O)—O—R_{f} with R_{f} as defined above, iii) C₁-C₆ (hydroxy)alkyl such as methyl or 2-hydroxyethyl;
d) a 5- or 6-membered noncationic heterocycle as defined previously, preferably chosen from morpholine, piperazine, piperidine, imidazole, pyrrole, pyrrolidine and oxazolidinone, said heterocycle preferably being unsubstituted;
e) a 5- or 6-membered cationic heterocycle as defined previously, preferably chosen from piperazinium and imidazolium, said heterocycle being optionally substituted with one or two C₁-C₄ alkyl radicals such as methyl;
f) an ammonium radical —N⁺RR′R″, An⁻, with R, R′ and R″, which may be identical or different, representing a (C₁-C₄)alkyl group such as methyl, preferably a trimethylammonium radical.

According to a preferred embodiment, the compounds of formula (I) are chosen from compounds (1) to (45) below, and also the geometrical or optical isomers thereof, the tautomers thereof, the organic or mineral acid or base salts thereof or the solvates thereof such as hydrates:

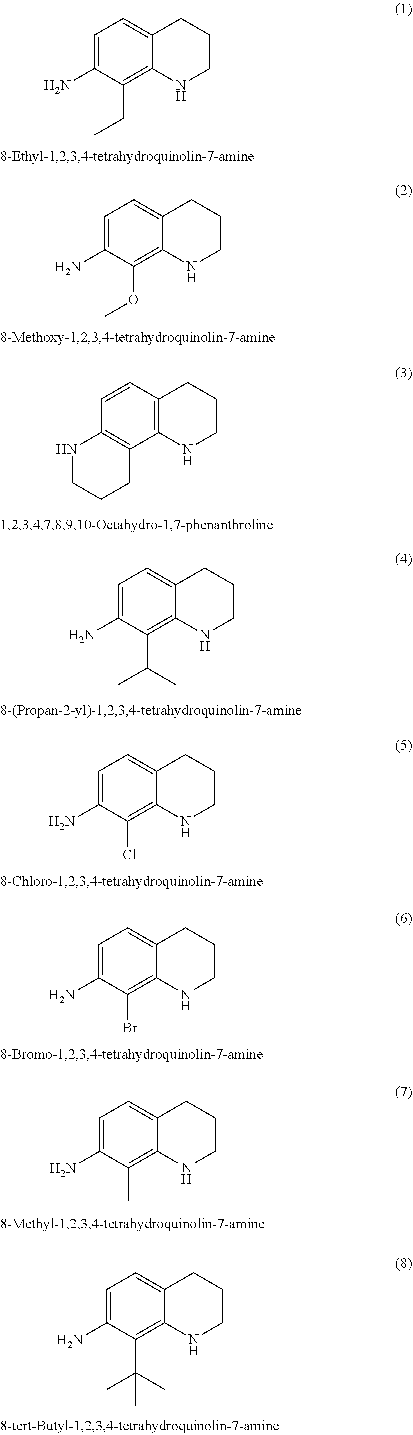

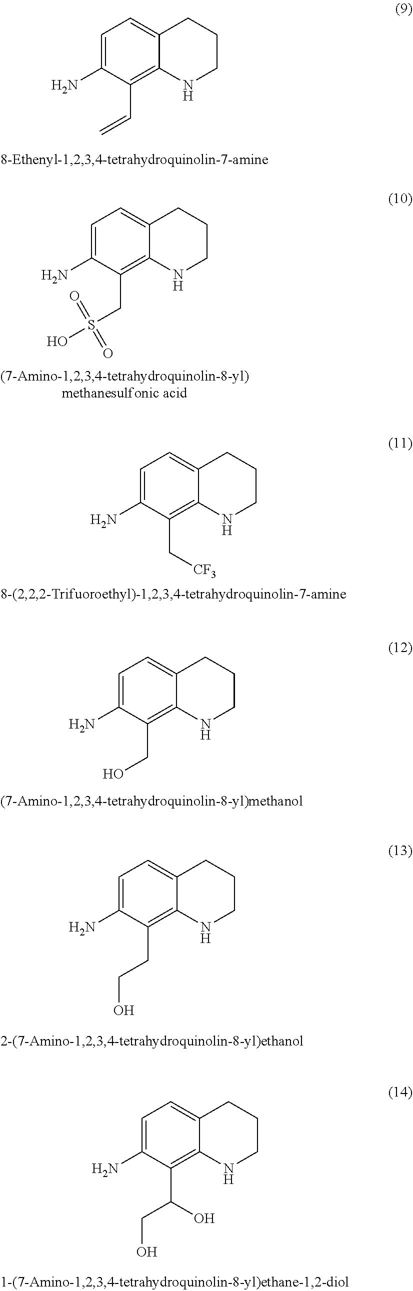 [SMILES: C=Cc1c(N)ccc2c1NCCC2 ; Nc1ccc2c(c1CS(=O)(=O)O)NCCC2 ; Nc1ccc2c(c1CC(F)(F)F)NCCC2 ; Nc1ccc2c(c1CO)NCCC2 ; Nc1ccc2c(c1CCO)NCCC2 ; Nc1ccc2c(c1C(O)CO)NCCC2]

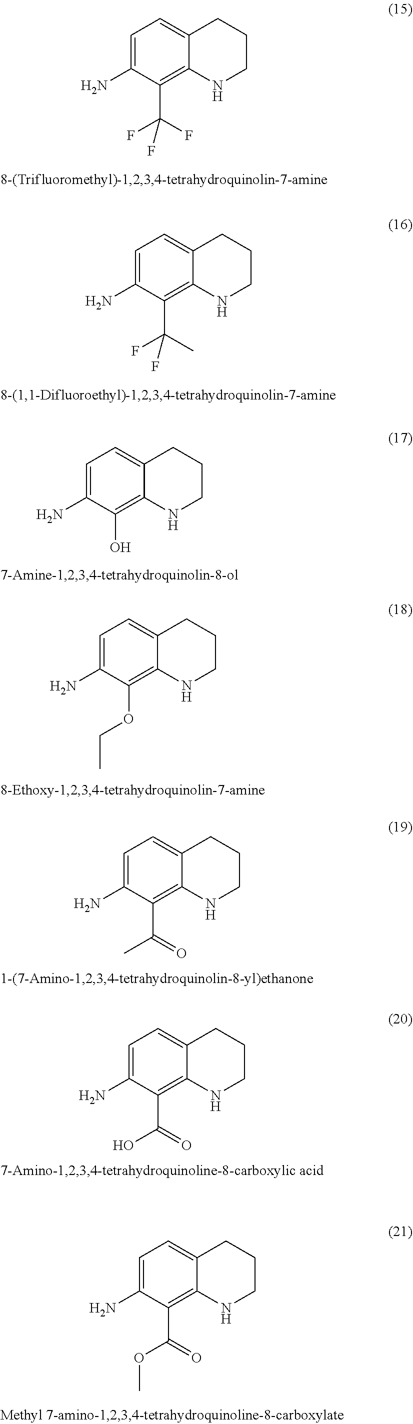

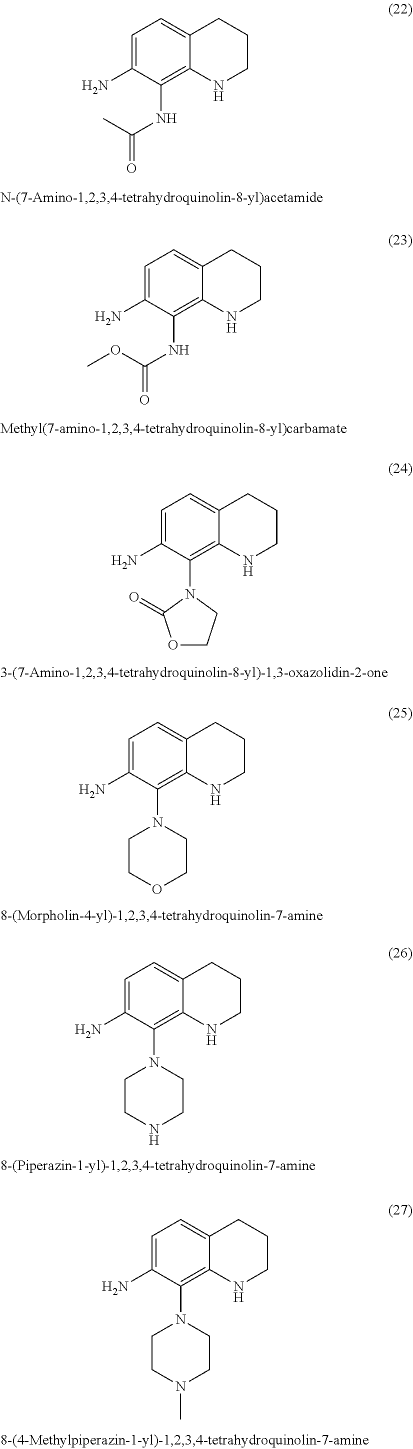 [SMILES: CC(=O)Nc1c(N)ccc2c1NCCC2 ; COC(=O)Nc1c(N)ccc2c1NCCC2 ; Nc1ccc2c(c1N1CCOC1=O)NCCC2 ; Nc1ccc2c(c1N1CCOCC1)NCCC2 ; Nc1ccc2c(c1N1CCNCC1)NCCC2 ; CN1CCN(c2c(N)ccc3c2NCCC3)CC1]

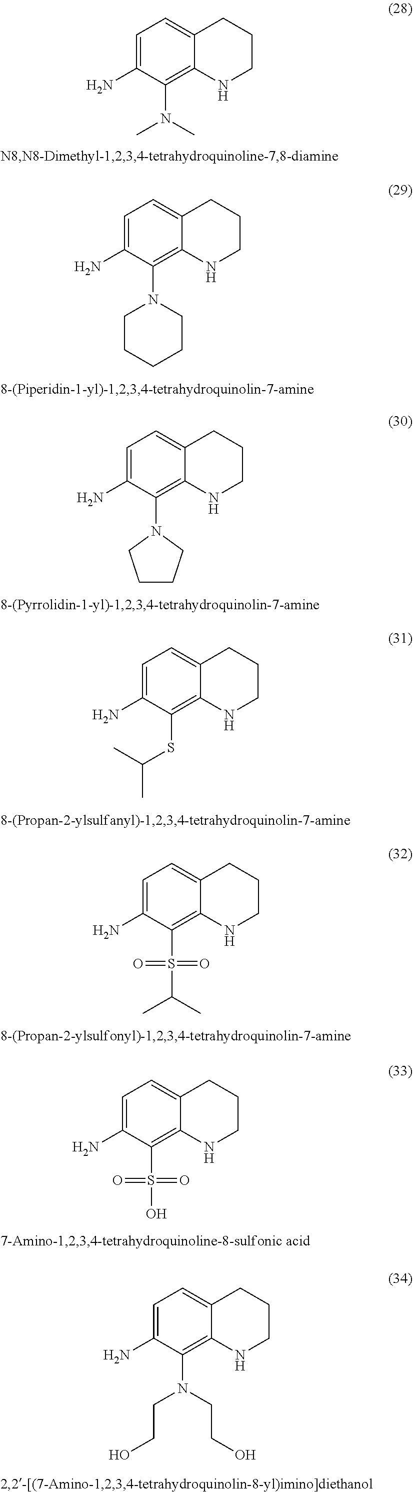

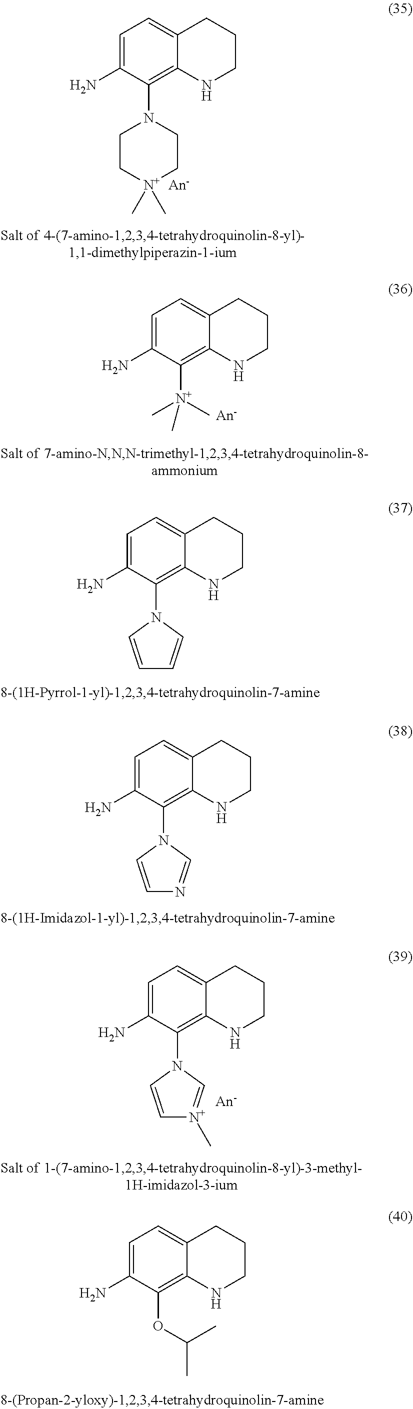 [SMILES: C[N+]1(C)CCN(c2c(N)ccc3c2NCCC3)CC1 ; C[N+](C)(C)c1c(N)ccc2c1NCCC2 ; Nc1ccc2c(c1-n1cccc1)NCCC2 ; Nc1ccc2c(c1-n1ccnc1)NCCC2 ; C[n+]1ccn(-c2c(N)ccc3c2NCCC3)c1 ; CC(C)Oc1c(N)ccc2c1NCCC2]

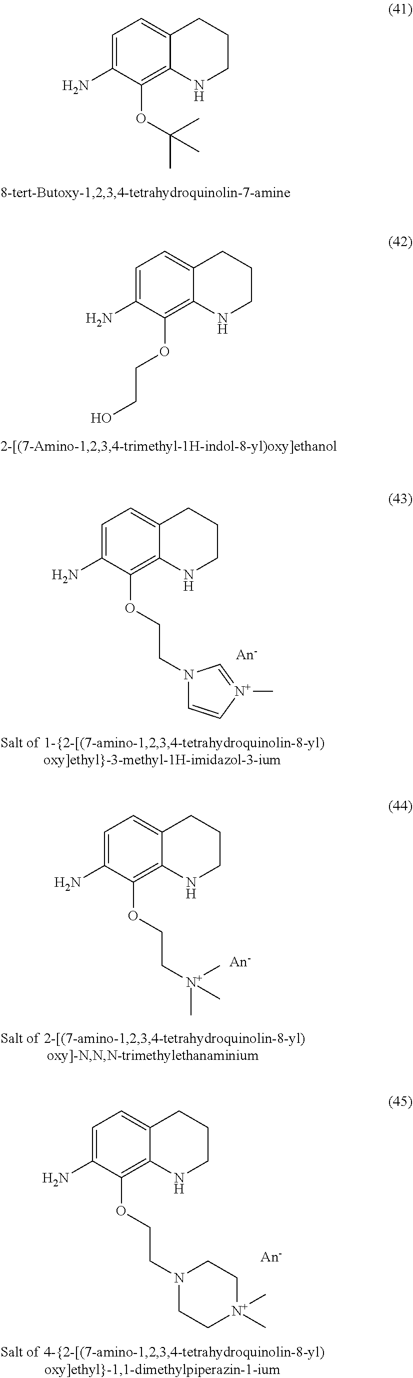 [SMILES: CC(C)(C)Oc1c(N)ccc2c1NCCC2 ; Nc1ccc2c(c1OCCO)NCCC2 ; C[n+]1ccn(CCOc2c(N)ccc3c2NCCC3)c1 ; C[N+](C)(C)CCOc1c(N)ccc2c1NCCC2 ; C[N+]1(C)CCN(CCOc2c(N)ccc3c2NCCC3)CC1]

with An⁻, which may be identical or different, representing an anion as defined previously, in particular chloride.

According to an advantageous form of the invention, the compounds are chosen from compounds (1), (2), (3), (25), (34) (37) and (39) above, and also the geometrical or optical isomers thereof, the tautomers thereof, the organic or mineral acid or base salts thereof or the solvates thereof such as hydrates.

Preferably, the process for dyeing keratin fibers, in particular human keratin fibers such as the hair, comprises at least one step i) of applying to said keratin fibers one or more compounds of formula (I) as defined previously and one or more oxidation bases as defined below; and optionally at least one step ii) of applying to said keratin fibers an oxidizing cosmetic composition comprising one or more chemical oxidizing agents, preferably chosen from hydrogen peroxide, urea peroxide, alkali metal bromates, persalts such as perborates and persulfates, peracids and oxidase enzymes (with the optional cofactors thereof), among which mention may be made of peroxidases, 2-electron oxidoreductases such as uricases and 4-electron oxygenases, for instance laccases; more preferentially, the chemical oxidizing agent is hydrogen peroxide.

According to this preference, when the step(s) ii) are performed, it is understood that between the step(s) i) and the step(s) ii), said fibers may be rinsed, and/or washed and then optionally dried.

Still according to this preference, the chemical oxidizing agent(s) are present in the oxidizing cosmetic composition in a total content of between 0.001% and 50% by weight, more preferentially between 0.05% and 30% by weight, even more preferentially between 0.1% and 20% by weight and even more particularly between 1% and 15% by weight relative to the total weight of the oxidizing cosmetic composition.

According to a preferred embodiment of the invention, the process for dyeing keratin fibers also comprises at least one step of applying one or more additional oxidation couplers as described below, other than the compounds of formula (I), and also the geometrical or optical isomers thereof, the tautomers thereof, the organic or mineral acid or base salts thereof or the solvates thereof according to the invention.

According to another preferred embodiment of the invention, the process for dyeing keratin fibers, in particular human keratin fibers such as the hair, also comprises at least one step ii) of applying to said keratin fibers an oxidizing cosmetic composition comprising one or more chemical oxidizing agents, preferably chosen from hydrogen peroxide, urea peroxide, alkali metal bromates, persalts such as perborates and persulfates, peracids and oxidase enzymes (with the optional cofactors thereof), among which mention may be made of peroxidases, 2-electron oxidoreductases such as uricases and 4-electron oxygenases, for instance laccases; more preferentially, the chemical oxidizing agent is hydrogen peroxide;

it being understood that between the step(s) i) and the step(s) ii), said fibers may be rinsed, and/or washed and then optionally dried.

Preferably, the compound(s) of formula (I) according to the invention are applied for a leave-on time of between 1 and 60 minutes, preferably between 5 and 40 minutes and even more preferentially between 10 and 30 minutes.

The compound(s) of formula (I) according to the invention are generally applied to the keratin fibers at room temperature, preferably between 25 and 55° C.

Preferably, steps i) and ii) are performed sequentially.

For the purposes of the present invention, the term “sequentially” means that the step(s) ii) are performed before or after the step(s) i), i.e. as a pretreatment or a post-treatment, preferably as a post-treatment.

According to one variant of the invention, the process according to the invention is a process for dyeing keratin fibers, in particular human keratin fibers such as the hair, comprising at least one step of simultaneous application to said fibers:
of one or more compounds of formula (I) as defined previously;
of one or more chemical oxidizing agents as defined previously;
optionally of one or more oxidation bases as described below.

According to this variant, a ready-to-use cosmetic composition may be prepared beforehand, resulting from the mixing of a cosmetic composition comprising one or more compounds of formula (I) as defined previously and optionally one or more oxidation bases, and of an oxidizing cosmetic composition comprising one or more chemical oxidizing agents as defined previously; in order subsequently to be applied to said fibers.

According to this variant of the invention, said ready-to-use cosmetic composition may be applied for a leave-on time of between 1 and 60 minutes, preferably between 5 and 40 minutes and even more preferentially between 10 and 30 minutes; and generally at room temperature, preferably between 25 and 55° C.

The term “simultaneous” means that the compound(s) of formula (I) as defined previously, the chemical oxidizing agent(s) as defined previously, and optionally the oxidation base(s) as described below, are applied at the same time to said keratin fibers.

Another subject of the invention relates to compounds of formula (I), and also the organic or mineral acid or base salts thereof, the tautomeric forms thereof, the optical isomers thereof, the geometrical isomers thereof and/or the solvates thereof such as the hydrates thereof, as described previously, in which:

A represents:
an iodine atom or a fluorine atom,
a carboxyl radical —COOH or carboxylate radical —COO⁻, M⁺,
a sulfonic radical —SO₃H or sulfonate radical —SO₃⁻M⁺, or alkylsulfonyl radical —S(O)₂—R with R representing a linear or branched (C₁-C₄)alkyl group,
a group —C(R₁₂)(R₁₃)—R₁₁ or —C(R₁₂)═C(R₁₁)—R₁₃, in which: R₁₁, R₁₂ and R₁₃, which may be identical or different, represent:
   a hydrogen atom,
   a halogen atom such as fluorine, chlorine, bromine or iodine, preferably fluorine,
   a hydroxyl radical,
   a linear or branched C₁-C₁₄ alkyl or alkenyl group, in particular C₁-C₈, preferably C₁-C₆, said alkyl or alkenyl group:
      optionally comprising one or more heteroatoms or groups, which may be identical or different, chosen from —O—, —S—, —S(O)—, —S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N⁼, ═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—NH—, and/or said alkyl or alkenyl group being
      optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) fluoro —F, vi) sulfonic —SO₃H, or sulfonate —SO₃⁻M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl; ix) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
it being understood that R₁₁, R₁₂ and R₁₃ cannot simultaneously denote a hydrogen atom,
   or R₁₁ forms with R₉, the nitrogen atom which bears R₉ and the carbon atoms which bear —N(R₉)(R₁₀) and R₁₁, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O—), it being understood that if R₁₀, R₁₂ and R₁₃ denote a hydrogen atom, R₉ and R₁₁ cannot constitute a divalent radical —(CH₂)₃—;
a group —W′ (R′₁₄) in which:
   W′ represents an oxygen or sulfur atom,
   R′₁₄ represents:
      a hydrogen atom,
      a linear or branched C₂-C₁₄ alkyl or alkenyl group, in particular C₂-C₈, preferably C₂-C₆, said alkyl or alkenyl group:
         optionally interrupted with one or more heteroatoms or groups, which may be identical or different, chosen from —O—, —S—, —S(O)—, —S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N═, ═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—N(H)—, and/or said alkyl or alkenyl group being
         optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) fluoro —F, vi) sulfonic —SO₃H, or sulfonate —SO₃⁻M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
      or R′₁₄ forms with R₉, the nitrogen atom which bears R₉ and the carbon atoms which bear —N(R₉)(R₁₀) and —W′—, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O⁻),
a group —N(R₁₅)—R′₁₅ in which R₁₅ and R′₁₅ independently represent:
   a hydrogen atom,
   a linear or branched C₁-C₁₄ alkyl or alkenyl group, in particular C₁-C₈, preferably C₁-C₆, said alkyl or alkenyl group:
      optionally interrupted with one or more heteroatoms or groups, which may be identical or different, chosen from —O—, —S—, —S(O)—, —S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N⁼, ═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—NH—, and/or said alkyl or alkenyl group being
      optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) fluoro —F, vi) sulfonic —SO₃H, or sulfonate —SO₃⁻M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl; ix) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
it being understood that R₁₅ and R′₁₅ cannot simultaneously denote a hydrogen atom;
   or R₁₅ forms with R₉, the nitrogen atom which bears R₉ and the carbon atoms which bear —N(R₉)(R₁₀) and —N(R′₁₅)—, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O⁻),
a carbonyl radical —C(O)—R₁₁, with R₁₁ as defined according to claim 1,
an ester radical —C(O)O—R₁₁, a thioester radical —C(O)S—R₁₁ or a dithioester radical —C(S)S—R₁₁, with R₁₁ as defined according to claim 1,
an optionally substituted cationic or noncationic (hetero)cycle,
an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
   with M⁺ as defined previously,
   and it being understood that the compounds of formula (I) are other than:
7-amino-8-fluoro-1,2,3,4-tetrahydroquinoline

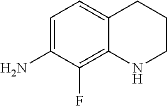 [SMILES: Nc1ccc2c(c1F)NCCC2]

7-amino-8-hydroxy-1,2,3,4-tetrahydroquinoline

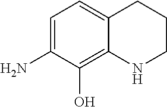 [SMILES: Nc1ccc2c(c1O)NCCC2]

According to a particular form of the invention, the novel compounds of formula (I) correspond to formula (IA) below, and also the geometrical or optical isomers thereof, the tautomers thereof, the organic or mineral acid or base salts thereof or the solvates thereof such as hydrates:

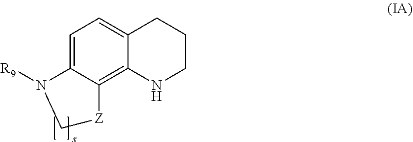

in which s denotes an integer equal to 1 or 2, Z denotes a radical C(R₁₂)(R₁₃) or a radical —N(R′₁₅) or a heteroatom chosen from O and S, and R₉, R₁₂, R₁₃ and R′₁₅ have the same definition as previously, it being understood that when s is equal to 2 and when Z denotes a CH₂ radical, then R₉ cannot denote a hydrogen atom.

According to another embodiment of the invention, the novel compounds of formula (I) denote the compounds of formula (IB) below, and also the geometrical or optical isomers thereof, the tautomers thereof, the organic or mineral acid or base salts thereof or the solvates thereof such as hydrates:

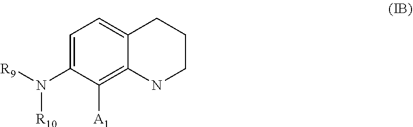

in which A₁ represents:
a) a radical —S—B or —SO₂—B in which B denotes a1) a hydrogen atom; a2) a C₁-C₆ alkyl or alkenyl radical, said alkyl or alkenyl radical being optionally substituted with one or more radicals independently chosen from: i) halogen such as fluorine, ii) hydroxyl, iii) SO₃H or SO₃⁻, M⁺ with M⁺ as defined previously, iv) ammonium —N⁺RR′R″, An⁻, with R, R′ and R″, which may be identical or different, representing a (C₁-C₄)alkyl group such as methyl, and in particular trimethylammonium; a3) a cationic heterocycle such as imidazolium or piperazinium;
b) a radical —O-T in which T denotes b1) a hydrogen atom; b2) a C₂-C₆ alkyl radical or a C₁-C₆ alkenyl radical, said alkyl or alkenyl radical being optionally substituted with one or more radicals chosen independently from: i) halogen such as fluorine, ii) hydroxyl, iii) SO₃H or SO₃⁻, M⁺ with M⁺ as defined previously, iv) ammonium —N⁺RR′R″, An⁻, with R, R′ and R″, which may be identical or different, representing a (C₁-C₄)alkyl group such as methyl, and in particular trimethylammonium, b3) a cationic heterocycle such as imidazolium or piperazinium;
c) a radical chosen from i) SO₃H or SO₃⁻, M⁺ with M⁺ as defined previously, ii) CO₂H or CO₂—, M⁺ with M⁺ as defined previously, iii) alkoxycarbonyl —CO₂R_{f} in which R_{f} denotes a C₁-C₄ alkyl radical such as methyl, iv) alkylcarbonyl —CO—R_{f} with R_{f} as defined above;
d) a radical —N(R′₁₄)R₁₄ with R′₁₄ and R₁₄ as defined previously and preferably with R′₁₄ denoting a hydrogen atom or a C₁-C₆ (hydroxy)alkyl radical such as methyl or 2-hydroxyethyl and R₁₄ denoting a radical chosen from i) alkylcarbonyl —C(O)—R_{f} with R_{f} as defined above, ii) alkoxycarbonyl —C(O)—O—R_{f} with R_{f} as defined above, iii) C₁-C₆ (hydroxy)alkyl such as methyl or 2-hydroxyethyl;
e) a noncationic heterocycle as defined previously, preferably chosen from morpholine, piperazine, piperidine, imidazole, pyrrole, pyrrolidine and oxazolidinone, said heterocycle preferably being unsubstituted;
f) a cationic heterocycle as defined previously, preferably chosen from piperazinium and imidazolium, said heterocycle being optionally substituted with one or two C₁-C₄ alkyl radicals such as methyl;
g) an ammonium radical —N⁺RR′R″, An⁻, with R, R′ and R″, which may be identical or different, representing a (C₁-C₄)alkyl group such as methyl, preferably a trimethylammonium radical.

According to a particular embodiment of the invention, the novel compounds of formula (I) or (IA) or (IB) are chosen from compounds (1), (4), (8), (9), (10), (11), (12), (13), (14), (15), (16), (18), (19), (20), (21), (22), (23), (24), (25), (26), (27), (28), (29), (30), (31), (32), (33), (34), (35), (36), (37), (38), (39), (40), (41), (42), (43), (44) and (45) and also the organic or mineral acid or base salts thereof, the tautomeric forms thereof, the optical isomers thereof, the geometrical isomers thereof and/or the solvates thereof such as the hydrates thereof.

According to a particular embodiment of the invention, the synthesis of the compounds of formula (I) for which the substituents R₁ to R₁₀ represent a hydrogen atom may be performed, for example, according to two synthetic strategies illustrated, respectively, by scheme (1) or (2) below.

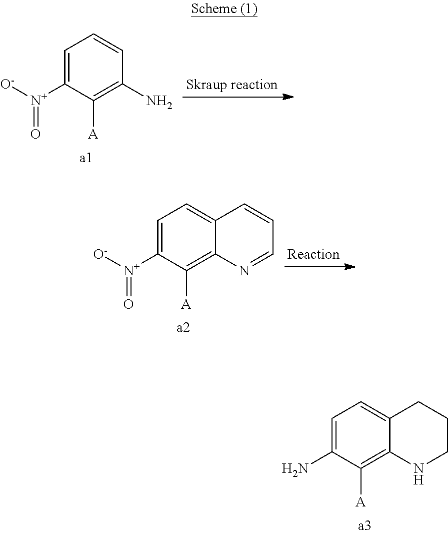

The first step of the first synthetic strategy illustrated by scheme (1) above consists of a Skraup reaction.

The Skraup reaction consists of the Michael addition of acrolein formed by glycerol in the presence of sulfuric acid, to the amine a1. An intramolecular electrophilic substitution, followed by dehydration on the intermediate thus formed, lead to the formation of the quinoline a2. This reaction may notably be performed in the presence of sodium iodide as catalyst.

Reduction of the nitro function of the intermediate a2 leads to the desired 7-aminotetrahydroquinoline a3.

The reduction step is performed under standard conditions known to those skilled in the art, preferably by catalytic reduction, for example by performing a hydrogenation reaction by heterogeneous catalysis in the presence of Pd/C, Pd(II)/C, Ni/Ra, etc., or alternatively by performing a reduction reaction with a metal, for example with zinc, iron, tin, etc. (see Advanced Organic Chemistry, 3rd Edition, J. March, 1985, Wiley Interscience and Reduction in Organic Chemistry, M. Hudlicky, 1983, Ellis Horwood Series Chemical Science).

The second synthetic strategy illustrated by scheme (2) below is broken down into three possible synthetic routes starting from 7-nitrotetrahydroquinoline.

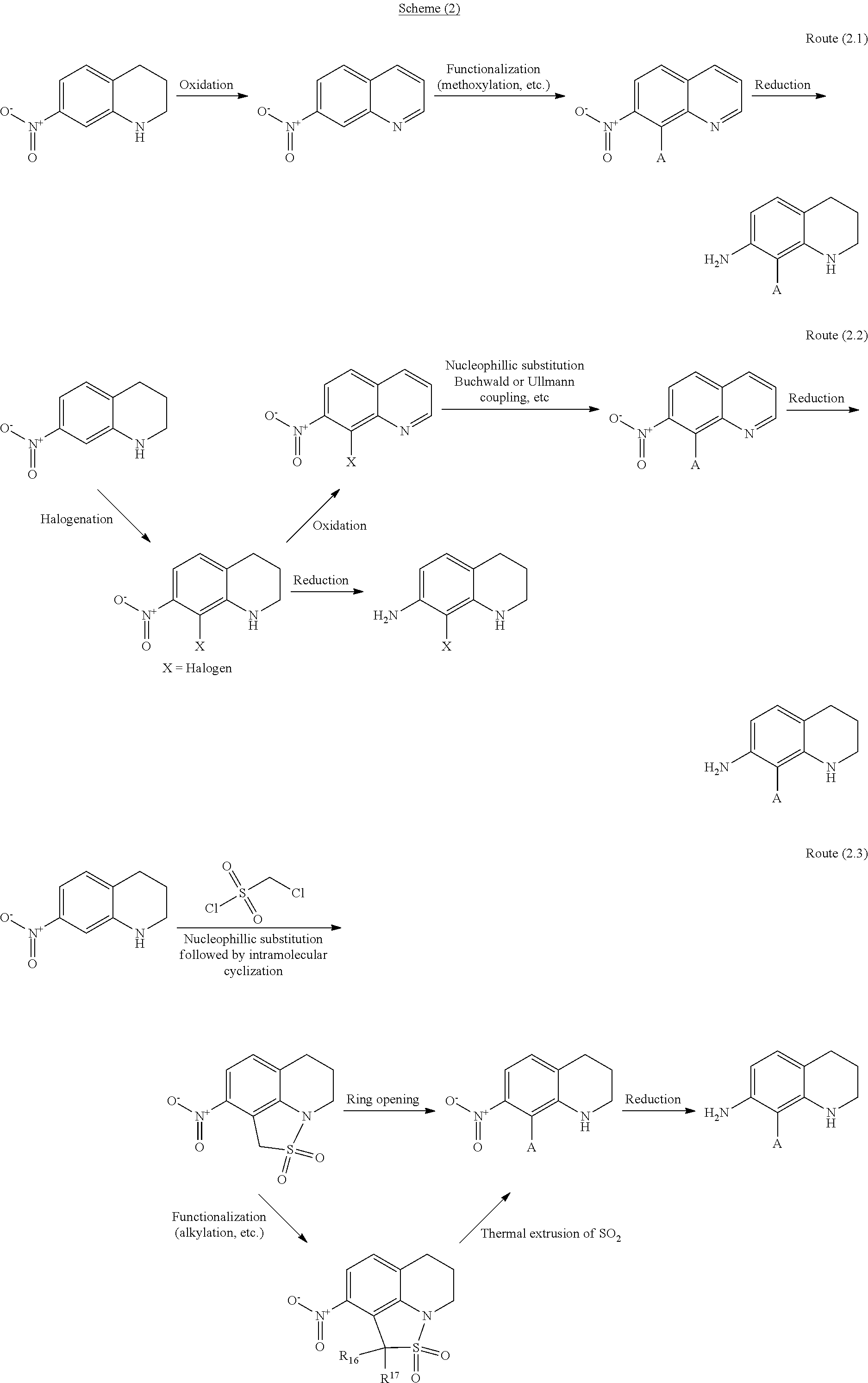

The first route (2.1) is notably used for the synthesis of 8-alkoxy-7-amino-1,2,3,4-tetrahydroquinoline compounds. This synthetic route consists of an oxidation, followed by functionalization, for example an O-alkylation, followed by a reduction to obtain the desired 8-substituted 7-amino-1,2,3,4-tetrahydroquinoline compound. This synthetic route is notably illustrated by the synthetic example 2 described below.

The second route (2.2) consists, in a first stage, of a step of halogenation of 7-nitrotetrahydroquinoline, for example by means of N-bromo- (or chloro)succinimide, followed by an oxidation, and then a nucleophilic substitution or Buchwald or Ullmann coupling, and finally a reduction to obtain the desired 8-substituted 7-amino-1,2,3,4-tetrahydroquinoline compound. This synthetic route is notably illustrated by the synthetic examples 4 to 7 described below.

Notably for the synthesis of compounds (5) and (6) as described previously, route (2.2) may also consist of the step of halogenation of 7-nitrotetrahydroquinoline, followed by a reduction step to give the desired 8-halo-7-amino-1,2,3,4-tetrahydroquinoline compounds.

The third route (2.3) consists of a nucleophilic substitution of 7-nitrotetrahydroquinoline with chloromethanesulfonyl chloride followed by an intramolecular cyclization. Next, the intermediate thus obtained:
is functionalized, notably an alkylation, on the carbon alpha to the SO₂ group and then undergoes a thermal extrusion of the SO₂ group to produce the 8-substituted 7-nitrotetrahydroquinoline intermediate; this synthetic route is notably illustrated by synthetic example 1 described below and compound (4) may also be produced via this route; or
undergoes opening of the dioxothiazolidine ring in basic medium to give the 8-substituted 7-nitrotetrahydroquinoline intermediate, which is useful, for example, for the synthesis of compound (10).

Finally, the 8-substituted 7-nitrotetrahydroquinoline intermediate is reduced to give the desired 8-substituted 7-aminotetrahydroquinoline product.

The reduction steps according to the second synthetic strategy are performed according to the same procedure as that described above for the first synthetic strategy.

The oxidation steps are performed under standard conditions known to those skilled in the art, preferably by reaction with 2,3-dichloro-5,6-dicyanobenzoquinone (DDQ) as oxidizing agent.

### Composition

The present invention also relates to a cosmetic composition comprising:
one or more compounds of formula (I) as defined previously; and
one or more oxidation bases.

Preferably, the compound(s) of formula (I) are chosen from compounds (1) to (45) as described previously.

A subject of the present invention is also a composition C2, notably a cosmetic composition C2, containing at least one compound of formula (IA) or of formula (IB or containing at least one compound chosen from compounds (1), (4), (8), (9), (10), (11), (12), (13), (14), (15), (16), (18), (19), (20), (21), (22), (23), (24), (25), (26), (27), (28), (29), (30), (31), (32), (33), (34), (35), (36), (37), (38), (39), (40), (41), (42), (43), (44) and (45) described previously, and also the organic or mineral acid or base salts thereof, the tautomeric forms thereof, the optical isomers thereof, the geometrical isomers thereof and/or the solvates thereof such as the hydrates thereof.

Preferably, the content of compound(s) of formula (I) or (IA) or (IB) in the composition according to the invention is between 0.001% and 20% by weight, more preferentially between 0.005% and 6% by weight relative to the total weight of the composition.

As indicated above, the cosmetic composition according to the invention also comprises at least one oxidation base.

By way of example, the oxidation bases are chosen from para-phenylenediamines, bis(phenyl)alkylenediamines, para-aminophenols ortho-aminophenols and heterocyclic bases, and the corresponding addition salts.

Among the para-phenylenediamines that may be mentioned are, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the corresponding addition salts with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the corresponding addition salts with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the corresponding addition salts.

Among the para-aminophenols that are mentioned are, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-β-hydroxyethylaminomethyl)phenol and 4-amino-2-fluorophenol, and the corresponding addition salts with an acid.

Among the ortho-aminophenols that may be mentioned are, for example, 2-aminophenol, 2-amino-5-methylphenol, 2-amino-6-methylphenol and 5-acetamido-2-aminophenol, and the corresponding addition salts.

Among the heterocyclic bases that may be mentioned are, for example, pyridine, pyrimidine and pyrazole derivatives.

Among the pyridine derivatives that may be mentioned are the compounds described, for example, in patents GB 1 026 978 and GB 1 153 196, for example 2,5-diaminopyridine, 2-(4-methoxyphenyl)amino-3-aminopyridine and 3,4-diaminopyridine, and the corresponding addition salts.

Other pyridine oxidation bases that are useful in the present invention are the 3-aminopyrazolo[1,5-a]pyridine oxidation bases or the corresponding addition salts described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, β-aminopyrazolo[1,5-a]pyrid-7-yl)methanol, 2-β-aminopyrazolo[1,5-a]pyrid-5-yl)ethanol, 2-β-aminopyrazolo[1,5-a]pyrid-7-yl)ethanol, β-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[β-aminopyrazolo[1,5-a]pyrid-5-yl)(2-hydroxyethyl)amino]ethanol, 2-[β-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-O-hydroxyethoxy-3-aminopyrazolo[1,5-a]pyridine; 2-(4-dimethylpiperazinium-1-yl)-3-aminopyrazolo[1,5-a]pyridine; and the corresponding addition salts.

More particularly, the oxidation bases that are useful in the present invention are chosen from 3-aminopyrazolo[1,5-a]pyridines and are preferably substituted on carbon atom 2 with:

a) a (di)(C₁-C₆)(alkyl)amino group, said alkyl group possibly being substituted with at least one hydroxyl, amino or imidazolium group;

b) an optionally cationic 5- to 7-membered heterocycloalkyl group comprising from 1 to 3 heteroatoms, optionally substituted with one or more (C₁-C₆)alkyl groups, such as a di(C₁-C₄)alkylpiperazinium group; or

c) a (C₁-C₆)alkoxy group optionally substituted with one or more hydroxyl groups, such as a 3-hydroxyalkoxy group, and the corresponding addition salts.

Among the pyrimidine derivatives that may be mentioned are the compounds described, for example, in patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-β-hydroxyethyl)amino-1-methylpyrazole, and the corresponding addition salts. Use may also be made of 4,5-diamino-1-(β-methoxyethyl)pyrazole.

A 4,5-diaminopyrazole will preferably be used, and even more preferentially 4,5-diamino-1-(β-hydroxyethyl)pyrazole and/or a corresponding salt.

The pyrazole derivatives that may also be mentioned include diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-β-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Use will preferably be made, as heterocyclic bases, of 4,5-diamino-1-(β-hydroxyethyl)pyrazole and/or 2,3-diamino-6,7-dihydro-1H,5H-pyrazolo[1,2-a]pyrazol-1-one and/or a corresponding salt.

The oxidation base(s) each advantageously represent from 0.001% to 10% by weight relative to the total weight of the composition, and preferably from 0.005% to 5% by weight relative to the total weight of the composition according to the invention.

Preferably, the cosmetic composition according to the invention also comprises at least one additional oxidation coupler, other than the compounds of formula (I), and also geometrical or optical isomers thereof, tautomers thereof, organic or mineral acid or base salts thereof or solvates thereof according to the invention.

Among these oxidation couplers, mention may be made in particular of meta-phenylenediamines, meta-aminophenols, meta-diphenols, naphthalene-based coupling agents and heterocyclic coupling agents, and also the corresponding addition salts.

Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-3-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole and 6-methylpyrazolo[1,5-a]benzimidazole, 2-methyl-5-aminophenol, 5-N-(β-hydroxyethyl)amino-2-methylphenol, 3-aminophenol and 3-amino-2-chloro-6-methylphenol, the corresponding addition salts with an acid and the corresponding mixtures.

Preferably, the additional oxidation coupler(s), other than the compounds of formula (I), and also the geometrical or optical isomers thereof, the tautomers thereof, the organic or mineral acid or base salts thereof or the solvates thereof according to the invention, if they are present, each advantageously represent 0.001% to 10% by weight, more preferentially 0.005% to 5% by weight, relative to the total weight of the composition according to the invention.

In general, the addition salts of oxidation bases and of couplers that may be used in the context of the invention are chosen in particular from the addition salts with an acid such as the hydrochlorides, hydrobromides, sulfates, citrates, succinates, tartrates, lactates, tosylates, benzenesulfonates, phosphates and acetates.

The composition according to the invention may optionally also comprise one or more synthetic or natural direct dyes, chosen from cationic, anionic and nonionic species, preferably cationic or nonionic species, either as sole additional dyes or in addition to the additional oxidation dye(s).

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (IIIa) and (III′a), the azo cationic dyes (IVa) and (IV′a) and the diazo cationic dyes (Va) below:

Het⁺-C(R^{a})═N—N(R^{b})—Ar, An⁻ (IIIa)

Het⁺-N(R^{a})—N═C(R^{b})—Ar, An⁻ (III′a)

Het⁺-N═N—Ar, An⁻ (IVa)

Ar⁺—N═N—Ar″, An⁻ (Va)

and

Het⁺-N═N—Ar′—N═N—Ar, An⁻ (IV′a)

in which formulae (IIIa), (III′a), (IVa), (IV′a) and (Va):
Het⁺ represents a cationic heteroaryl radical, preferably bearing an endocyclic cationic charge, such as imidazolium, indolium or pyridinium, which is optionally substituted, preferably with one or more (C₁-C₈) alkyl groups such as methyl;
Ar⁺ represents an aryl radical, such as phenyl or naphthyl, bearing an exocyclic cationic charge, preferably ammonium, in particular tri(C₁-C₈)alkylammonium, such as trimethylammonium;
Ar represents an aryl group, in particular phenyl, which is optionally substituted, preferably with one or more electron-donating group such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or, as a variant, Ar represents a julolidine group;
Ar′ represents an optionally substituted divalent (hetero)arylene group such as phenylene, particularly para-phenylene, or naphthalene, which is optionally substituted, preferably with one or more (C₁-C₈)alkyl, hydroxyl or (C₁-C₈)alkoxy groups;
Ar″ represents an optionally substituted (hetero)aryl group, such as phenyl or pyrazolyl, which is optionally substituted, preferably with one or more (C₁-C₅)alkyl, hydroxyl, (di)(C₁-C₈)(alkyl)amino, (C₁-C₈)alkoxy or phenyl groups;
R^{a} and R^{b}, which may be identical or different, represent a hydrogen atom or a (C₁-C₈)alkyl group, which is optionally substituted, preferably with a hydroxyl group;

or, as a variant, the substituent R^{a} with a substituent of Het⁺ and/or R^{b} with a substituent of Ar and/or R^{a} with R^{b} form, together with the atoms that bear them, a (hetero)cycloalkyl;

in particular, R^{a} and R^{b} represent a hydrogen atom or a (C₁-C₄)alkyl group, which is optionally substituted with a hydroxyl group;
An⁻ represents an anionic counterion, such as mesylate or halide.

Mention may be made in particular of the azo and hydrazono cationic dyes bearing an endocyclic cationic charge of formulae (IIIa), (III′a) and (IVa) as defined previously. More particularly those of formulae (IIIa), (III′a) and (IVa) derived from the dyes described in patent applications WO 95/15144, WO 95/01772 and EP-714954.

Preferably, the cationic part is derived from the following derivatives:

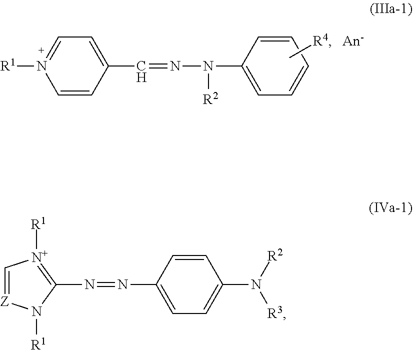

formulae (IIIa-1) and (IVa-1) with:
R¹ representing a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino group optionally substituted on the alkyl group(s) with a hydroxyl group; in particular, R⁴ represents a hydrogen atom;
Z represents a CH group or a nitrogen atom, preferentially CH;
An⁻ represents an anionic counterion, such as mesylate or halide.

In particular, the dye of formulae (IIIa-1) and (IVa-1) is chosen from Basic Red 51, Basic Yellow 87 and Basic Orange 31 or corresponding derivatives:

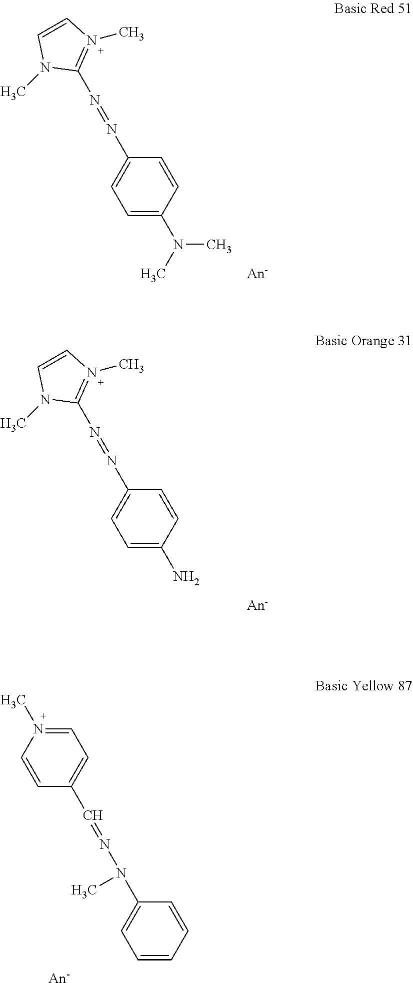

Among the natural direct dyes that may be used according to the invention, mention may be made of hennotannic acid, juglone, alizarin, purpurin, carminic acid, kermesic acid, purpurogallin, protocatechaldehyde, indigo, isatin, curcumin, spinulosin, apigenidin and orcein. Extracts or decoctions containing these natural dyes and in particular henna-based poultices or extracts may also be used.

When they are present, the direct dye(s) more particularly represent from 0.001% to 10% by weight and preferably from 0.005% to 5% by weight relative to the total weight of the composition.

According to a preferred embodiment of the invention, the cosmetic composition according to the invention comprises:
one or more groups of formula (I) as defined previously; preferably, the compounds of formula (I) are chosen from compounds (1) to (45) as defined previously, more preferentially from compounds (1), (4), (8), (9), (10), (11), (12), (13), (14), (15), (16), (18), (19), (20), (21), (22), (23), (24), (25), (26), (27), (28), (29), (30), (31), (32), (33), (34), (35), (36), (37), (38), (39), (40), (41), (42), (43), (44) and (45) as defined previously;
one or more oxidation bases; and
one or more chemical oxidizing agents.

Preferably, the chemical oxidizing agents are chosen from hydrogen peroxide, urea peroxide, alkali metal bromates, persalts such as perborates and persulfates, peracids, and oxidase enzymes (with the possible cofactors thereof), among which mention may be made of peroxidases, 2-electron oxidoreductases such as uricases, and 4-electron oxygenases, for instance laccases; preferably, the chemical oxidizing agent is hydrogen peroxide.

Preferably, when they are present, the total content of the chemical oxidizing agent(s) present in the cosmetic composition according to the invention is between 0.001% and 30% by weight, more preferentially between 0.05% and 20% by weight and even more preferentially between 0.2% and 15% by weight, relative to the total weight of the cosmetic composition.

The pH of the oxidizing composition containing the chemical oxidizing agent(s) is such that, after mixing with the cosmetic composition, the pH of the resulting composition applied to the keratin fibers preferably ranges between 2 and 12 approximately, even more preferentially between 3 and 10 and even more particularly between 4 and 9.5. It may be adjusted to the desired value by means of acidifying or basifying agents usually used in the dyeing of keratin fibers and as defined previously.

According to another variant of the invention, the cosmetic composition is a ready-to-use cosmetic composition, notably for dyeing keratin fibers, in particular human keratin fibers such as the hair, which results from the mixing of a cosmetic composition comprising one or more compounds of formula (I) or (IA) or (IB) as defined previously and of an oxidizing composition comprising one or more chemical oxidizing agents.

According to another variant of the invention, the composition comprising one or more compounds of formula (I) or (IA) or (IB) as defined previously is free of chemical oxidizing agent.

Preferably, the composition also comprises a cosmetically acceptable medium for the dyeing of keratin fibers.

The term “cosmetically acceptable medium” means a medium that is suitable for dyeing keratin fibers, which generally comprises water or a mixture of water and at least one organic solvent, for instance branched or unbranched C₁-C₄ lower alcohols, such as ethanol and isopropanol; polyols and polyol ethers, such as 2-butoxyethanol, propylene glycol, propylene glycol monomethyl ether, diethylene glycol monoethyl ether and monomethyl ether, and glycerol, and also aromatic alcohols such as benzyl alcohol or phenoxyethanol, and mixtures thereof.

The pH of the composition according to the invention may be adjusted to the desired value by means of one or more acidifying agents and/or one or more basifying agents.

Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

Among the basifying agents, examples that may be mentioned include aqueous ammonia, alkali metal carbonates, alkanolamines, such as mono-, di- and triethanolamines and derivatives thereof, sodium hydroxide, potassium hydroxide and the compounds of formula (VI) below: RₐR_{b}N—Z—NR_{c}R_{d}; in which Z is a linear or branched (C₁-C₆)alkylene group, optionally substituted notably with one or more hydroxyl or amino groups; preferably, Z=propylene optionally substituted with a hydroxyl group or a C₁-C₄ alkyl radical; Rₐ, R_{b}, R_{c} and R_{d}, which may be identical or different, represent a hydrogen atom or a C₁-C₄ alkyl or C₁-C₄ hydroxyalkyl radical.

Advantageously, the cosmetic composition according to the invention may also comprise at least one additive chosen from fragrances, surfactants (cationic, anionic, nonionic or amphoteric), sequestrants, polymers, ceramides, silicones, preserving agents, nacreous agents or opacifiers, and vitamins or provitamins.

When they are present, the total content of the additive(s) present in the composition is between 0.01% and 20% by weight relative to the total weight of the composition according to the invention.

The cosmetic composition according to the invention may be in various forms, such as in the form of liquids, creams, gels or foams, or in any other form that is suitable for dyeing keratin fibers, and notably human hair.

A subject of the invention is also a process for dyeing keratin fibers, in particular human keratin fibers such as the hair, comprising at least one step of applying to said keratin fibers the cosmetic composition according to the invention as defined previously, and optionally at least one step of applying to said keratin fibers an oxidizing cosmetic composition as described previously.

According to this embodiment, when the step(s) ii) are performed, it is understood that between the step(s) i) and the step(s) ii), said fibers may be rinsed, and/or washed and then optionally dried.

A subject of the present invention is also a multi-compartment device, or dyeing “kit”, comprising a first compartment containing one or more compounds of formula (I) as defined previously, and a second compartment comprising one or more chemical oxidizing agents as defined previously.

According to a preferred embodiment of the invention, the multi-compartment device according to the invention comprises a first compartment containing the cosmetic composition according to the invention as defined previously, and a second compartment comprising an oxidizing cosmetic composition containing one or more oxidizing chemical agents as defined previously.

According to a variant of the invention, the device according to the invention comprises a compartment containing a ready-to-use cosmetic composition as defined previously.

The examples that follow serve to illustrate the invention without, however, being limiting in nature.

The compounds were fully characterized via standard spectroscopic or spectrometric methods known to those skilled in the art.

### EXAMPLES OF SYNTHESIS

### Examples 1: Synthesis of 8-ethyl-1,2,3,4-tetrahydroquinolin-7-amine dihydrochloride (See Synthesis, 6 (1992) 571-576 for the First Two Steps)

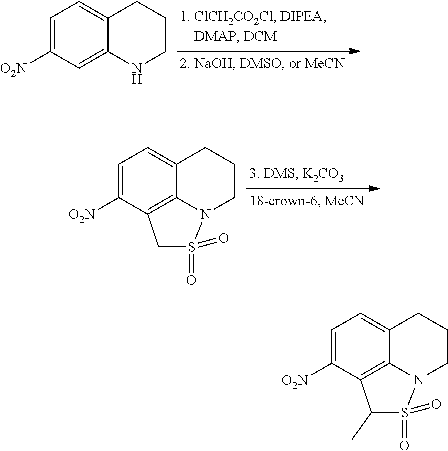 [SMILES: O=[N+]([O-])c1ccc2c(c1)NCCC2 ; O=[N+]([O-])c1ccc2c3c1CS(=O)(=O)N3CCC2 ; CC1c2c([N+](=O)[O-])ccc3c2N(CCC3)S1(=O)=O]

1. 6.06 g of 7-nitrotetrahydroquinoline and 417 mg of 4-dimethylaminopyridine are placed in 23 ml of N,N-diisopropylethylamine in a 250 ml round-bottomed flask equipped with a magnetic bar. While cooling, a solution of 3.0 ml of chloromethanesulfonyl chloride in 34 ml of anhydrous dichloromethane is added dropwise to the reaction medium and the mixture is then left stirring at room temperature for 5 hours. Saturated ammonium chloride solution is then added slowly to the reaction medium to quench the reaction. After shaking and separation of the two phases, the aqueous phase is extracted three times with dichloromethane. The combined organic phases are washed with water and then with saturated sodium chloride solution, and then dried over anhydrous sodium sulfate and concentrated under reduced pressure. The residue is purified by flash chromatography on a column of silica gel (eluent: 20/80 ethyl acetate/heptane) to give the expected substituted product obtained in the form of a dark yellow powder.
2. 2.6 g of 1-chloromethanesulfonyl-7-nitro-1,2,3,4-tetrahydroquinoline are placed in 14 ml of dimethyl sulfoxide in a 100 ml round-bottomed flask equipped with a magnetic bar, this solution is added dropwise to another flask which contains 5.16 g of sodium hydroxide in 38 ml of dimethyl sulfoxide, and the reaction medium is then left stirring at room temperature for 70 minutes. The reaction medium is then added to a mixture of 20 ml of ammonium chloride and 100 ml of dichloromethane. The aqueous phase is extracted three times with dichloromethane and the combined organic phases are washed with water and then with saturated sodium chloride solution. After drying over anhydrous sodium sulfate, the solvent is evaporated off under reduced pressure and the residue is purified by standard flash column chromatography (eluent: 30/70 ethyl acetate/heptane). After removing the solvent, 1.57 g of the cyclized product are obtained in the form of a yellow powder.
3. 502 mg of the cyclized intermediate, 2.06 g of potassium carbonate and 7.1 mg of 18-crown-6 (0.01 eq.) are placed in 12 ml of acetonitrile in a 50 ml round-bottomed flask equipped with a magnetic bar. 0.11 ml of dimethyl sulfate is then added dropwise to the solution obtained, with stirring, and the reaction medium is then refluxed for 4 hours. The reaction medium is then added with stirring to a mixture of water and ethyl acetate. The aqueous phase is also extracted three times with dichloromethane and the combined organic phases are washed with water and then with saturated sodium chloride solution. After drying over anhydrous sodium sulfate, the solvent is evaporated off under reduced pressure and the residue is purified by standard flash column chromatography (eluent: 35/65 ethyl acetate/heptane). The methylated tricycle is then obtained in the form of a brown solid.

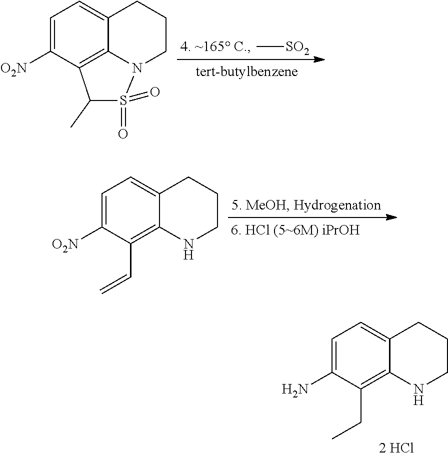

4. 0.88 g of the methylated tricycle is placed in 25 mL of tert-butylbenzene in a 100 ml round-bottomed flask equipped with a magnetic bar, and the reaction medium is then maintained at 165° C. for 22 hours. The reaction medium is purified directly by standard flash column chromatography (eluent: 20/80 dichloromethane/heptane and then 15/85 ethyl acetate/heptane) to give the expected 7-nitro-8-vinyl-1,2,3,4-tetrahydroquinoline intermediate in the form of an orange-yellow oil.
5. Hydrogenation is then performed in an H-cube on 0.46 g of 7-nitro-8-vinyl-1,2,3,4-tetrahydroquinoline under the following conditions: Concentration: 0.01 mol/L in methanol, Pressure: 40 bar, Temperature: 70° C., Hydrogen production: 90%, Cartridge: 10% Pd/C, Flow rate: 3 mL/min.
6. In the last step, the product is salified with 12 ml of hydrochloric acid solution (5-6M) in 200 ml of isopropanol, the solution is then concentrated and the final product dried. 8-Ethyl-1,2,3,4-tetrahydroquinolin-7-amine dihydrochloride is thus obtained in the form of a beige-colored powder.

### Examples 2: Synthesis of 8-methoxy-1,2,3,4-tetrahydroquinolin-7-amine dihydrochloride (see J. Chem. Soc., Perkin Trans. 1, (2000) 1259-1264 for the Second Step)

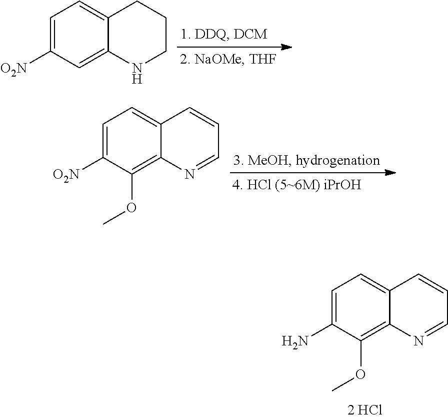

1. 10 g of 7-nitroquinoline are placed in 770 ml of dichloromethane in a 1 L three-necked round-bottomed flask equipped with a thermometer and a magnetic bar, followed by addition of 26 g of 2,3-dichloro-5,6-dicyano-1,4-benzoquinone. After stirring for 4 hours, the reaction medium is filtered and the filtrate is then evaporated under vacuum and then dried under vacuum. The product is obtained in the form of an orange powder.
2. 9.9 g of 7-nitroquinoline are placed in 220 ml of tetrahydrofuran in a 1 L round-bottomed flask equipped with a thermometer and a magnetic bar, followed by dropwise addition of 100 ml of sodium methoxide. After stirring for 18 hours at room temperature, water is added and the mixture is then neutralized to pH 7. The resulting mixture is filtered under vacuum and the filtrate is then concentrated on a rotary evaporator and 200 ml of ethyl acetate are added. After shaking and separation of the two phases, the aqueous phase is extracted four times with ethyl acetate. The combined organic phases are washed twice with water and once with saturated sodium chloride solution. After drying over anhydrous magnesium sulfate, the organic phase is concentrated under reduced pressure. The crude product is obtained in the form of a brown powder, which is purified by standard flash column chromatography (eluent: 25/75 ethyl acetate/heptane) to give 8-methoxy-7-nitroquinoline in the form of yellow crystals.
3. A reduction is then performed with an H-cube on the product obtained in the preceding step under the following conditions: Concentration: 0.02 mol/L in methanol, Pressure: 60 bar, Temperature: 70° C., Hydrogen production: 80%, Cartridge: 10% Pd/C, Flow rate: 3 mL/min.
4. The reduced product is then salified in an identical manner to that of step 6 of example 1. 8-Methoxy-1,2,3,4-tetrahydroquinolin-7-amine dihydrochloride is obtained in the form of a white powder.

### Examples 3: Synthesis of 1,2,3,4,7,8,9,10-octahydro-1,7-phenanthroline dihydrochloride (see Monatshefte für Chemie, 119 (1988) 761-780 for the First Step)

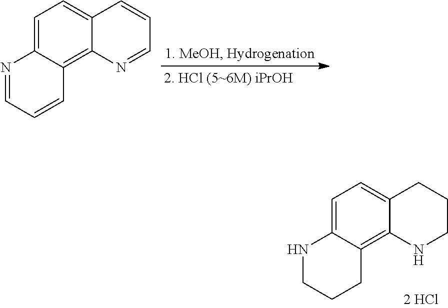 [SMILES: c1cnc2c(c1)ccc1ncccc12 ; c1cc2c(c3c1CCCN3)CCCN2]

1. A reduction is performed with an H-cube starting with 1,7-phenanthroline under the following conditions: Concentration: 0.02 mol/L in methanol, Pressure: 60 bar, Temperature: 70° C., Hydrogen production: 80%, Cartridge: 10% Pd/C, Flow rate: 3 mL/min.
2. The reduced product is then salified in an identical manner to that of step 6 of example 1. Recrystallization is then performed after evaporating off the excess acid. The product is obtained in the form of a white powder.

### Examples 4: Synthesis of 8-(morpholin-4-yl)-1,2,3,4-tetrahydroquinolin-7-amine dihydrochloride

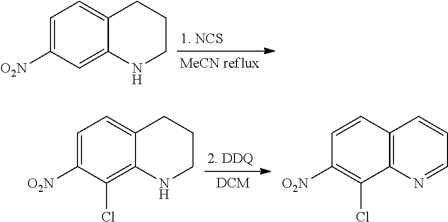 [SMILES: O=[N+]([O-])c1ccc2c(c1)NCCC2 ; O=[N+]([O-])c1ccc2c(c1Cl)NCCC2 ; O=[N+]([O-])c1ccc2cccnc2c1Cl]

1. The synthesis is performed starting with 7-nitrotetrahydroquinoline. 15 g of 7-nitrotetrahydroquinoline are placed in 300 ml of acetonitrile in a 500 ml three-necked round-bottomed flask equipped with a thermometer and a magnetic bar, followed by addition of 11.3 g of N-chlorosuccinimide. The reaction medium is then refluxed for 4 hours 30 minutes. The medium is transferred into a 1 L round-bottomed flask containing 50 ml of water and 300 ml of ethyl acetate. The mixture is concentrated on a rotary evaporator and 200 ml of ethyl acetate and 100 ml of water are then added. After stirring and separation of the two phases, the aqueous phase is extracted twice with ethyl acetate. The combined organic phases are washed twice with water and once with saturated sodium chloride solution. After drying over anhydrous magnesium sulfate, the organic phase is concentrated under reduced pressure. Purification by standard flash column chromatography (eluent: 10/90 dichloromethane/heptane) is then performed, and the chloro product is obtained in the form of an orange-colored powder.
2. 650 mg of 8-chloro-7-nitrotetrahydroquinoline are placed with stirring in 25 ml of dichloromethane in a 100 ml three-necked round-bottomed flask equipped with a thermometer and a magnetic bar. 1.38 mg of 2,3-dichloro-5,6-dicyano-1,4-benzoquinone are then added. After stirring for 6 hours, the reaction medium is filtered and the filtrate is evaporated under vacuum on a rotary evaporator. The product is purified by standard flash column chromatography (eluent: 30/70 ethyl acetate/heptane) and 8-chloro-7-nitroquinoline is obtained in the form of a yellow powder.

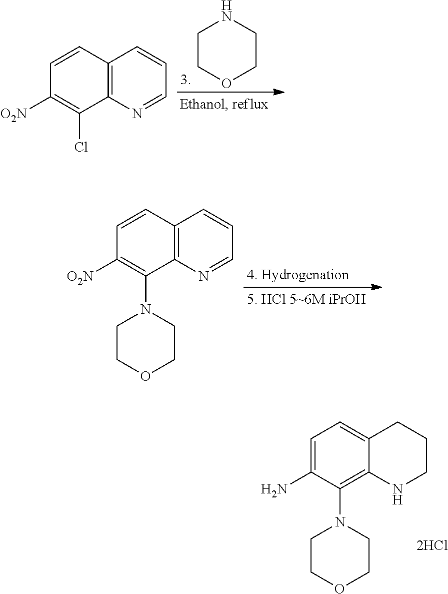

3. 500 mg of the intermediate obtained previously are placed in 25 ml of ethanol in a 100 ml three-necked round-bottomed flask equipped with a thermometer and a magnetic bar, and 630 μl of morpholine are added. The mixture is then refluxed for 8 hours and the reaction medium is then evaporated to dryness under vacuum. 100 ml of ethyl acetate and 100 ml of water are added. After stirring and separation of the two phases, the aqueous phase is extracted with ethyl acetate. The combined organic phases are washed twice with water and then once with saturated sodium chloride solution. After drying over anhydrous magnesium sulfate, the organic phase is concentrated under reduced pressure. Purification by standard flash column chromatography (eluent: 10/90 ethyl acetate/heptane) is then performed, and 610 mg of the expected product are thus obtained.
4. A reduction is then performed with an H-cube under the following conditions: Concentration: 0.02 mol/L in methanol, Pressure: 60 bar, Temperature: 70° C., Hydrogen production: 80%, Cartridge: 10% Pd/C, Flow rate: 3 mL/min.
5. The reduced product is then salified in an identical manner to that of step 6 of example 1. 200 mg of 8-(morpholin-4-yl)-1,2,3,4-tetrahydroquinolin-7-amine dihydrochloride are obtained in the form of a white powder.

### Examples 5: Synthesis of 2,2′-[(7-amino-1,2,3,4-tetrahydroquinolin-8-yl)imino]diethanol dihydrochloride

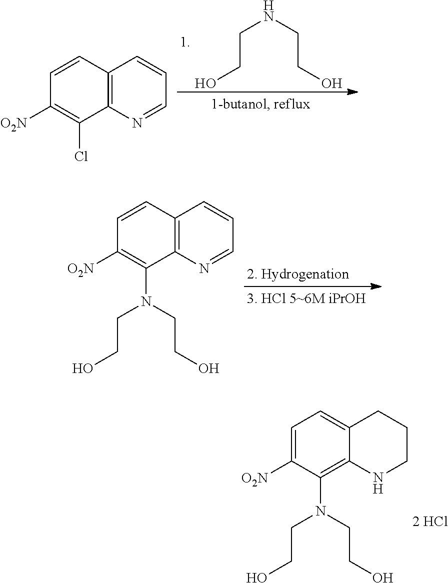

1. The synthesis is performed starting with the intermediate 8-chloro-7-nitroquinoline, as described in the synthesis of example 4. 13.0 g of 8-chloro-7-nitroquinoline are placed in 500 ml of 1-butanol in a 1 L three-necked round-bottomed flask equipped with a thermometer and a magnetic bar. 18 ml of diethanolamine are then added to the solution obtained, with stirring, and the reaction medium is then refluxed for 16 hours. After evaporating off the solvent under vacuum, dichloromethane and water are added to the residue. After shaking and separation of the two phases, the aqueous phase is extracted three times with dichloromethane. The combined organic phases are washed three times with water and then once with saturated sodium chloride solution. After drying over anhydrous magnesium sulfate, the organic phase is concentrated under reduced pressure and then taken up in diethyl ether and the mixture is stirred for 2 hours until an orange precipitate appears. After removing the solvent, the nitro precursor of the expected product is isolated in the form of an orange-colored powder.
2. 7.0 g of 8-diethanolamine-7-nitroquinoline and 0.7 g of Pd/C (10% by weight) are placed in 250 ml of ethanol in a 1 L autoclave. The autoclave is then sealed and purged with nitrogen, and then filled with 20 bar of H₂. After stirring for 48 hours at room temperature, the reaction medium is filtered through Celite, the solvent is then removed under reduced pressure and the residue is purified by standard flash column chromatography (eluent: 10/90 methanol/dichloromethane).
3. The product is then salified with 19 ml of hydrochloric acid solution in isopropanol.
After filtration and removal of the solvent, the expected product is isolated in the form of a light brown powder.

### Examples 6: Synthesis of 1-(7-amino-1,2,3,4-tetrahydroquinolin-8-yl)-3-methyl-1H-imidazol-3-ium chloride hydrochloride

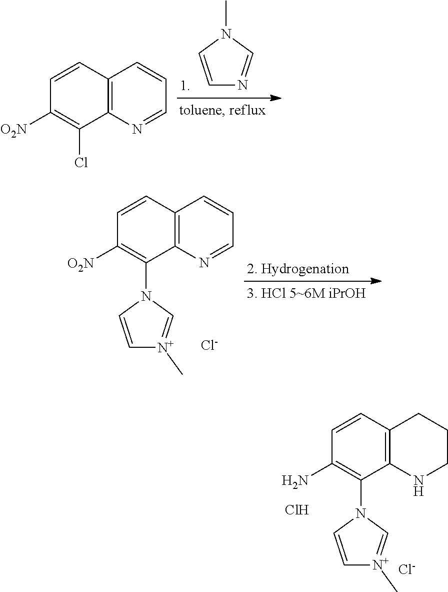

1. The synthesis is performed starting with the intermediate 8-chloro-7-nitroquinoline, as described in the synthesis of example 4. 7.0 g of 8-chloro-7-nitroquinoline are placed in 250 ml of toluene in a 500 ml three-necked round-bottomed flask equipped with a thermometer and a magnetic bar. 8 ml of 1-methylimidazole are then added to the solution obtained, with stirring, and the reaction medium is then refluxed for 16 hours. A brown precipitate appears, which is filtered off and then washed with diethyl ether. The precipitate is then taken up in acetonitrile and the mixture is refluxed for 10 minutes. After cooling to room temperature, the precipitate obtained is filtered off to give the nitro precursor of the expected product in the form of a light brown powder.
2. 6.0 g of 8-(1-methylimidazolium)-7-nitroquinoline chloride and 0.6 g of Pd/C (10% by weight) are placed in 250 ml of ethanol in a 1 L autoclave. The autoclave is then sealed and purged with nitrogen, and then filled with 20 bar of H₂. Since the reaction is incomplete after 3 days of stirring at room temperature, a further 0.6 g of Pd/C (10% by weight) is added and the reduction is continued for a further 2 days. The reaction medium is filtered through Celite, the solvent is then removed under reduced pressure and the residue is purified by standard flash column chromatography (eluent: 20/80 methanol/dichloromethane).
3. The product is then salified with 3 eq. of hydrochloric acid solution in isopropanol. After removal of the solvent, the 1-(7-amino-1,2,3,4-tetrahydroquinolin-8-yl)-3-methyl-1H-imidazol-3-ium chloride hydrochloride is isolated in the form of a pale yellow powder.

### Examples 7: Synthesis of 8-(1H-pyrrol-1-yl)-1,2,3,4-tetrahydroquinolin-7-amine hydrochloride

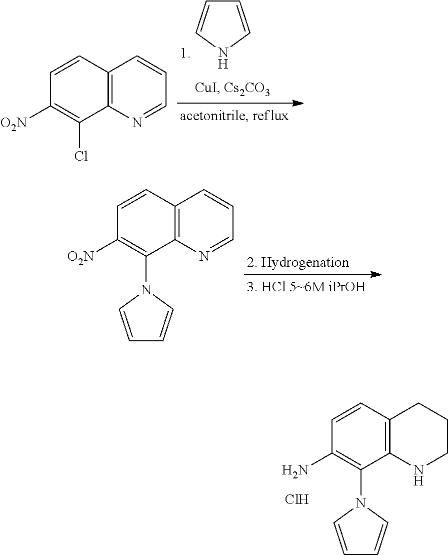 [SMILES: c1cc[nH]c1 ; O=[N+]([O-])c1ccc2cccnc2c1Cl ; O=[N+]([O-])c1ccc2cccnc2c1-n1cccc1 ; Cl ; Nc1ccc2c(c1-n1cccc1)NCCC2]

1. The synthesis is performed starting with the intermediate 8-chloro-7-nitroquinoline, as described in example 4. 15 g of 8-chloro-7-nitroquinoline are placed in 150 ml of acetonitrile in a 500 ml three-necked round-bottomed flask equipped with a thermometer and a magnetic bar. 25 ml of pyrrole and 70 g of cesium carbonate are then added to the solution obtained, with stirring. The flask is then purged with nitrogen, and 1.36 g of copper (I) iodide are then added. The reaction medium is refluxed under nitrogen for 16 hours, and the reaction is monitored by liquid-phase chromatography coupled to mass spectrometry. The reaction medium is then filtered through silica and eluted with ethyl acetate, the filtrate is then concentrated under reduced pressure and the residue is purified by standard flash column chromatography (eluent: 20/80 ethyl acetate/petroleum ether) to give 8-pyrrole-7-nitroquinoline.
2. 7.3 g of 8-pyrrole-7-nitroquinoline and 0.73 g of Pd/C (10% by weight) are placed in 250 ml of ethanol in a 1 L autoclave. The autoclave is then sealed and purged with nitrogen, and then filled with 15 bar of H₂. After stirring for 4 days at room temperature, since the reaction is incomplete, 0.6 g of Pd/C (10% by weight) is added and the reaction is continued under 20 bar of H₂ for 3 days. The reaction medium is then filtered through Celite, the solvent is then removed under reduced pressure and the residue is purified twice by standard flash column chromatography (eluent: 65/35 dichloromethane/petroleum ether).
3. The product is then salified with 3 eq. of hydrochloric acid solution in isopropanol. After removal of the solvent, the 8-(1H-pyrrol-1-yl)-1,2,3,4-tetrahydroquinolin-1-amine hydrochloride is isolated in the form of a yellow powder.

### DYEING EXAMPLES

### Example 1: Dyeing Evaluation of the Couplers

a) Composition

Composition (A) according to the present invention was prepared using the ingredients of which the contents, expressed as mass percentages of active material relative to the total weight of the composition, are indicated in the table below.

| | Composition A |
|---|---|
| 96° ethyl alcohol | 20.8 |
| Aqueous 35% sodium metabisulfite solution | 0.23 |
| Pentasodium salt of diethylenetriaminepentaacetic | 0.48 |
| acid as an aqueous 40% solution | |
| C₈-C₁₀ alkyl poly glucoside as an aqueous 60% | 3.6 |
| solution | |
| Benzyl alcohol | 2.0 |
| Polyethylene glycol containing 8 units of ethylene | 3.0 |
| oxide | |
| NH₄C1 | 4.32 |
| Aqueous ammonia containing 20% NH₃ | 2.94 |
| Coupler | 10⁻³ mol |
| Oxidation base | 10⁻³ mol |
| Water | qs 100 |

Composition (A) was prepared with each of the oxidation bases below.

| Oxidation base | Composition |
|---|---|
| para-Aminophenol | A1 |
| 4-Aminophenylamine dihydrochloride | A2 |
| 2-Methylbenzene-1,4-diamine sulfate | A3 |
| 2-[(4-Aminophenyl)(2-hydroxyethyl)amino]ethanol sulfate | A4 |
| 3-(2,5-Diaminophenyl)propan-1-ol dihydrochloride | A5 |
| 2-(4,5-Diamino-lH-pyrazol-1-yl)ethanol sulfate | A6 |
| 2,3-Diamino-6,7-dihydro-1H,5H-pyrazolo[1,2- | A7 |
| a]pyrazol-1-one dimethanesulfonate | |
| 2-[(3-Aminopyrazolo[1,5-a]pyridin-2- | A8 |
| yl)oxy]ethanol hydrochloride | |
| 4-(3-Aminopyrazolo[1,5-a]pyridin-2-yl)- | A9 |
| 1,1-dimethylpiperazin-1-ium chloride hydrochloride | |

Seven series (S1), (S2), (S3), (S4), (S5), (S6) and (S7) of compositions (A1) to (A9) were thus prepared.

Each of the compositions (A1) to (A9) of series (S1) comprises 8-ethyl-1,2,3,4-tetrahydroquinolin-7-amine dihydrochloride as coupler.

Each of the compositions (A1) to (A9) of series (S2) comprises 8-methoxy-1,2,3,4-tetrahydroquinolin-7-amine dihydrochloride as coupler.

Each of the compositions (A1) to (A9) of series (S3) comprises 1,2,3,4,7,8,9,10-octahydro-1,7-phenanthroline dihydrochloride as coupler.

Each of the compositions (A2) to (A9) of series (S4) comprises 8-(morpholin-4-yl)-1,2,3,4-tetrahydroquinolin-7-amine dihydrochloride as coupler.

Each of the compositions (A2) to (A9) of series (S5) comprises 2,2′-[(7-amino-1,2,3,4-tetrahydroquinolin-8-yl)imino]diethanol dihydrochloride as coupler.

Each of the compositions (A1) to (A9) of series (S6) comprises 1-(7-amino-1,2,3,4-tetrahydroquinolin-8-yl)-3-methyl-1H-imidazol-3-ium hydrochloride as coupler.

Each of the compositions (A2) to (A9) of series (S7) comprises 8-(1H-pyrrol-1-yl)-1,2,3,4-tetrahydroquinolin-7-amine hydrochloride as coupler.

b) Procedure

At the time of use, each of the compositions (A1) to (A9) is mixed with 20-volumes aqueous hydrogen peroxide solution (6% by weight) in a 1:1 ratio. The final pH of each of the mixtures is equal to 9.5.

Each mixture thus obtained is applied to a lock of gray hair containing 90% white hairs. After a leave-on time of 30 minutes, the locks are rinsed, washed with a standard shampoo and then rinsed again. The locks are then dried.
c) Results

The color results are given in the tables below:

Summary of the dyeing evaluations for (S1) to (S7)

| | S1 | S2 | S3 | S4 | S5 | S6 | S7 |
|---|---|---|---|---|---|---|---|
| A1 | Red | Red | Red | — | — | Matt yellow | — |
| A2 | Blue | Blue | Blue | Blue | Blue | Green | Blue-green |
| A3 | Blue | Blue | Blue | Blue | Blue | Green | Blue-green |
| A4 | Blue-green | Blue-green | Blue-green | Blue-green | Green-blue | Green | Green |
| A5 | Blue | Blue | Blue-green | Blue | Blue | Green | Green |
| A6 | Red-violet | Violet-red | Red-violet | Red-violet | Red-violet | Red-violet | Violet-red |
| A7 | Red | Red | Red | Red | Coppery red | Red | Mahogany red |
| A8 | Violet-red | Violet-red | Violet-red | Violet | Violet | Violet | Violet |
| A9 | Green | Green | Green | Green | Green | Green | Green |

### Example 2: Evaluation of the Persistence with Respect to Shampooing and to Light, and the Chromaticity of the Dyeing Results

The intensity of the coloring obtained for each of the compositions was evaluated in the CIE L* a* b* system, using a Minolta Spectrophotometer CM3610D colorimeter. In this L* a* b* system, the three parameters respectively denote the intensity of the color (L*), the green/red color axis (a*) and the blue/yellow color axis (b*).

The comparative evaluation was performed with the same dye support (A) as described in example 1 and under the same operating conditions.

The couplers are numbered as follows:
C1 is 8-ethyl-1,2,3,4-tetrahydroquinolin-7-amine dihydrochloride (Invention);
C2 is 8-methoxy-1,2,3,4-tetrahydroquinolin-7-amine dihydrochloride (Invention);
C3 is 1,2,3,4,7,8,9,10-octahydro-1,7-phenanthroline dihydrochloride (Invention);
C4 is 8-(morpholin-4-yl)-1,2,3,4-tetrahydroquinolin-7-amine dihydrochloride (Invention);
C5 is 2,2′-[(7-amino-1,2,3,4-tetrahydroquinolin-8-yl)imino]diethanol dihydrochloride (Invention);
C6 is 1-(7-amino-1,2,3,4-tetrahydroquinolin-8-yl)-3-methyl-1H-imidazol-3-ium chloride hydrochloride (Invention);
C7 is 8-(1H-pyrrol-1-yl)-1,2,3,4-tetrahydroquinolin-7-amine hydrochloride (Invention); and
D is 1,2,3,4-tetrahydroquinolin-7-amine dihydrochloride (Comparative).

The oxidation bases are numbered as follows:
B1 is 4-aminophenylamine dihydrochloride;
B2 is 2-methylbenzene-1,4-diamine sulfate; and
B3 is 4-aminophenol.

Evaluation of the Persistence with Respect to Shampooing of the Dyeing Results

At the time of use, each of the compositions is mixed with 20-volumes aqueous hydrogen peroxide solution (6% by weight) in a 1:1 ratio. The final pH of each of the mixtures is equal to 9.5.

Each mixture thus obtained is applied to a lock of gray hair containing 90% white hairs. After a leave-on time of 30 minutes, the locks are rinsed, washed with a standard shampoo and then rinsed again. The locks are then dried.

The difference in color build-up before and after cycles of one shampoo wash and three rinses were calculated by applying the formula below:

DE=\sqrt{(L−L*)²+(a−a*)²+(b−b*)²}

L*, a* and b*, as defined previously, are the values measured before shampooing but after dyeing; L, a and b are the values measured after dyeing and after shampooing (DOP˜50%, 6 cycles of 1 shampoo wash +3 rinses, on a Pall transparent plate) or (DOP˜2% 6 shampoo washes).

| | Bases | | |
|---|---|---|---|
| Couplers | B1 | B2 | B3 |
| D (10⁻³ mol) comparative | 7.76 | 8.15 | 11.43 |
| C1 (10⁻³ mol) | 2.36 | 1.65 | 6.35 |
| C2 (10⁻³ mol) | — | 3.19 | — |
| C4 (10⁻³ mol) | 3.34 | 4.49 | 5.31 |
| C5 (10⁻³ mol) | 4.02 | 4.39 | 6.57 |
| C6 (10⁻³ mol) | 4.72 | 4.32 | 3.34 |
| C7 (10⁻³ mol) | 4.67 | 5.00 | 5.14 |

Evaluation of the Persistence with Respect to Light of the Dyeing Results

At the time of use, each of the compositions is mixed with 20-volumes aqueous hydrogen peroxide solution (6% by weight) in a 1:1 ratio. The final pH of each of the mixtures is equal to 9.5.

Each mixture thus obtained is applied to a lock of gray hair containing 90% white hairs. After a leave-on time of 30 minutes, the locks are rinsed, washed with a standard shampoo and then rinsed again. The locks are then dried.

The difference in color build-up-up before and after exposure to light was calculated by applying the formula below:

DE=\sqrt{(L−L*)²+(a−a*)²+(b−b*)²}

L*, a* and b* as defined previously, are the values measured before exposure to light but after dyeing; L′, a′ and b′ are the values measured after dyeing and after exposure to light (Xenon-1600W, exposure for 2 h 26 min, equivalent to 1 month of normal life).

Bases

| Couplers | B1 | B3 |
|---|---|---|
| D (10⁻³ mol) comparative | 10.55 | 11.51 |
| C1 (10⁻³ mol) | 4.41 | 6.44 |
| C2 (10⁻³ mol) | 6.19 | 6.60 |
| C3 (10⁻³ mol) | 3.41 | 4.62 |
| C5 (10⁻³ mol) | 6.66 | — |
| C6 (10⁻³ mol) | 5.25 | 3.92 |

Evaluation of the Chromaticity of the Dyeing Results

At the time of use, each of the compositions is mixed with 20-volumes aqueous hydrogen peroxide solution (6% by weight) in a 1:1 ratio. The final pH of each of the mixtures is equal to 9.5.

Each mixture thus obtained is applied to a lock of gray hair containing 90% white hairs. After a leave-on time of 30 minutes, the locks are rinsed, washed with a standard shampoo and then rinsed again. The locks are then dried.

The chromaticity of the color after dyeing was calculated by applying the formula below:

c*=\sqrt{a*²+b*²}

a* and b* as defined previously, are the values measured after dyeing.

Bases

| Couplers | B1 | B2 |
|---|---|---|
| D (10⁻³ mol) | 8.71 | 8.99 |
| C1 (10⁻³ mol) | 11.96 | 12.96 |
| C2 (10⁻³ mol) | 13.58 | 12.73 |
| C3 (10⁻³ mol) | 10.8 | 13.15 |

It is seen from the above results of the dyeing process according to the invention which uses couplers of 7-aminotetrahydroquinoline type substituted in position 8, relative to the dyeing process which uses a coupler of 7-aminotetrahydroquinoline type not substituted in position 8, that the colorings obtained with the process of the invention are significantly more persistent with respect to shampooing and to light and more chromatic than the comparative.

### Example 3: Comparative Evaluation of the Build-Up, the Intensity and the Chromaticity of the Dyeing Results

The comparative evaluation was performed with the dye compositions prepared from the following ingredients:

| Compositions | Amount | |
|---|---|---|
| Oxidation base | 0.05 | mmol |
| Oxidation coupler | 0.05 | mmol |
| Water | 7.0 | ml |
| Ethanol | 2.0 | mL |
| Aqueous ammonia containing 20% NH₃ | 1.0 | mL |
| 20-volumes aqueous hydrogen peroxide | 1.0 | mL |
| solution | | |

Each composition obtained is applied to 1 g locks of natural Caucasian hair containing 90% white hairs. After a leave-on time of 30 minutes at 27° C., the locks are rinsed, washed with a standard shampoo, rinsed again and then dried.

The oxidation couplers are numbered as follows:
C2 is 8-methoxy-1,2,3,4-tetrahydroquinolin-7-amine dihydrochloride (Invention);
D2 is 8-methoxy-1,2,3,4-tetrahydroquinolin-5-amine (Comparative)

The oxidation bases are numbered as follows:
A6 is 2-(4,5-diamino-1H-pyrazol-1-yl)ethanol sulfate;
A7 is 2,3-diamino-6,7-dihydro-1H,5H-pyrazolo[1,2-a]pyrazol-1-one dimethanesulfonate; and
A8 is 2-[β-aminopyrazolo[1,5-a]pyridin-2-yl)oxy]ethanol hydrochloride.

### L, A, b Results:

The colorimetric data for each of the locks are then measured with a Minolta CM-3610d spectrophotometer. In this L* a* b* system, L* represents the lightness, a* indicates the green/red color axis and b* indicates the blue/yellow color axis. The higher the value of L, the lighter or less intense the color. Conversely, the lower the value of L, the darker or more intense the color. The higher the value of a*, the redder the shade, and the higher the value of b*, the yellower the shade.
The color build-up on hair thus corresponds to the variation in coloring between the locks of dyed NG hair (natural gray hair containing 90% white hairs) and the non-dyed (i.e. untreated) NG hair, which is measured by (ΔE) according to the following equation:

ΔE=\sqrt{(L−L₀*)²+(a−a₀*)²+(b−b₀*)²}

In this equation, L*, a* and b* represent the values measured after dyeing of the NG hair, and L₀*, a₀* and b₀* represent the values measured before dyeing of the NG hair. The higher the ΔE value, the better the build-up of the coloring.

Evaluation of the Build-Up of the Dyeing Results (ΔE)

Couplers

| | D2 | C2 |
|---|---|---|
| Bases | (Comparative) | (Invention) |
| A6 | 43.76 | 55.68 |
| A8 | 35.32 | 49.18 |

Evaluation of the Intensity of the Dyeing Results (L*)

Couplers

| | D2 | C2 |
|---|---|---|
| Bases | (Comparative) | (Invention) |
| A8 | 33.13 | 21.04 |

The chromaticity is calculated according to the following formula:

C*=\sqrt{(a*)²+(b*)²}

The higher the chromaticity value C*, the more chromatic the color of the treated keratin fibers.

Evaluation of the Chromaticity of the Dyeing Results (C*)

Couplers

| | D2 | C2 |
|---|---|---|
| Bases | (Comparative) | (Invention) |
| A6 | 20.22 | 31.59 |
| A7 | 24.56 | 32.49 |

It is seen from the above results that the colorings obtained with the dyeing process according to the invention using couplers of 7-aminotetrahydroquinoline type substituted in position 8 are significantly more intense, more chromatic and with a better color build-up than the colorings obtained via a comparative dyeing process using couplers of 5-aminotetrahydroquinoline type substituted in position 8.

1. A process for dyeing keratin fibers, in particular human keratin fibers such as the hair, comprising at least one step i) of applying to said keratin fibers one or more compounds of formula (I) below, and also the organic or mineral acid or base salts thereof, the tautomeric forms thereof, the optical isomers thereof, the geometrical isomers thereof and/or the solvates thereof such as the hydrates thereof: 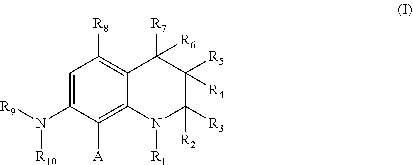
in which formula (I):
A represents:
i) a halogen atom,
ii) a carboxyl radical —COOH or carboxylate radical —COO⁻M⁺,
iii) a sulfonic radical —SO₃H or sulfonate radical —SO₃⁻M⁺, or alkylsulfonyl radical —S(O)₂—R with R representing a linear or branched (C₁-C₄)alkyl group,
iv) a group —C(R₁₂)(R₁₃)—R₁₁ or —C(R₁₂)═C(R₁)—R₁₃, in which R₁₁, R₁₂ and R₁₃, which may be identical or different, represent:
a hydrogen atom,
a halogen atom such as fluorine, chlorine, bromine or iodine, preferably fluorine,
a hydroxyl radical,
a linear or branched C₁-C₁₄ alkyl or C₂-C₁₄ alkenyl, in particular C₁-C₈ alkyl or C₂-C₈ alkenyl, preferably C₁-C₆ alkyl or C₂-C₆ alkenyl group, said alkyl or alkenyl group:
optionally interrupted with one or more heteroatoms or groups, which may be identical or different, chosen from —O—, —S—, —S(O)—, —S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—,
—C(O)—N═, ═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—N(H)—,
and/or said alkyl or alkenyl group being
optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) halo such as fluoro —F, vi) sulfonic —SO₃H, or sulfonate —SO₃-M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl; ix) an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl;
or else R₁₁ forms with R₉, the nitrogen atom which bears R₉ and the carbon atoms which bear —N(R₉)—R₁₀ and R₁₁, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O—),
v) a group —W—R₁₄ in which:
W represents an oxygen or sulfur atom or a divalent group chosen from:
N(R′₁₄)—,—S(O)— and —S(O)₂—,
R₁₄ and R′₁₄ independently represent:
a hydrogen atom,
a linear or branched C₁-C₁₄ alkyl or C₂-C₁₄ alkenyl group, in particular C₁-C₈, preferably C₁-C₆, said alkyl or alkenyl group being:
optionally interrupted with one or more heteroatoms or groups, which may be identical or different, chosen from —O—, —S—, —S(O)—,
—S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N═, ═N—C(O)—, —C(O)—N(H)—, —N(H)—C(O)—, —N(H)—C(O)—N(H)—, —O—C(O)—N(H)—, —N(H)—C(O)—O—,
—N(H)—C(NH)—N(H)—,
and/or said alkyl or alkenyl group being optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) halo such as fluoro —F, vi) sulfonic —SO₃H, or sulfonate —SO₃-M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl; ix) an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl;
or else R₁₄ forms with R₉, the nitrogen atom which bears R₉ and the carbon atoms which bear —N(R₉)(R₁₀) and —W—, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O⁻),
vi) a radical —C(X′)—R₁₁, preferably carbonyl —C(O)—R₁₁, with R₁₁ as defined previously,
vii) a radical —C(X′)—X″—R₁₁, preferably ester —C(O)—O—R₁₁, thioester —C(O)—S—R₁₁, dithioester —C(S)—S—R₁₁, with R₁₁ as defined previously, —X″—C(X′)—R₁₁, preferably amide —N(R′)—C(O)—R₁₁ or —X″—C(X′)—X—R₁₁, preferably carbamate —N(R′)—C(O)—O—R₁₁,
with X as defined previously, and X′ and X″, which may be identical or different, represent an oxygen or sulfur atom or a group N(R′) with R′ representing a hydrogen atom or a C₁-C₄ alkyl group; preferably, X′ represents an oxygen atom and X″ represents an oxygen or sulfur atom or a group N(R′) with R′ representing a hydrogen atom or a C₁-C₄ alkyl group;
viii) an optionally substituted cationic or noncationic (hetero)cycle,
ix) an ammonium radical —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl,
R₁ represents:
a hydrogen atom,
a saturated or unsaturated, linear or branched C₁-C₆ alkyl radical, said alkyl radical:
optionally comprising one or more heteroatoms or groups, which may be identical or different, chosen from —O—, —S—, —S(O)—, —S(O)₂—, —N(H)—,
—N(R)—, ═N—R—, —R—N═ and —C(X)— with R as defined previously, and with X representing an oxygen or sulfur atom or N—R, or combinations of said heteroatoms or groups, preferably chosen from: —O—C(O)—, —C(O)—O—,
—C(O)—N(H)—, —N(H)—C(O)—, —C(O)—N═, ═N—C(O)—, —N(H)—C(O)—N(H)—, —O—C(O)—N(H)—, —N(H)—C(O)—O—, —N(H)—C(NH)—NH—, and/or said alkyl radical being
optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) cationic or noncationic (hetero)cycle;
R₂, R₃, R₄, R₅, R₆ and R₇, which may be identical or different, represent:
a hydrogen atom,
a hydroxyl radical,
a saturated or unsaturated, linear or branched C₁-C₆ alkyl radical, said alkyl radical:
optionally comprising one or more heteroatoms or groups, which may be identical or different, chosen from —O—, —S—, —S(O)—, —S(O)₂—, —N(H)—, —N(R)—, ═N—R—, —R—N═ and —C(X)— with R as defined previously, and with X representing an oxygen or sulfur atom or N—R, or combinations of said heteroatoms or groups, preferably chosen from: —O—C(O)—,
—C(O)—O—, —C(O)—N(H)—, —N(H)—C(O)—, —C(O)—N═, ═N—C(O)—, —N(H)—C(O)—N(H)—, —O—C(O)—N(H)—, —N(H)—C(O)—O—, —N(H)—C(NH)—NH—, and/or said alkyl radical being
optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino,
an aromatic or nonaromatic, saturated or unsaturated, 5- to 8-membered (hetero)cycle, optionally substituted with one or more radicals, which may be identical or different, chosen from the following radicals: i) hydroxyl, ii) C₁-C₆ (hydroxy)alkyl, iii) C₁-C₆ (hydroxy)alkoxy, iii) amino —NH₂, iv) C₁-C₆ mono and/or di(hydroxy)alkylamino, and at least one of the members of which may represent a divalent oxo radical;
R₈ represents:
a hydrogen atom,
a halogen atom such as fluorine, chlorine, bromine or iodine, preferably chlorine, fluorine or bromine,
a hydroxyl radical,
a (C₁-C₈)alkylcarbonyl radical,
a saturated or unsaturated, linear or branched C₁-C₈ alkyl radical, said alkyl radical:
optionally comprising one or more heteroatoms or groups, which may be identical or different, chosen from —O—, —S—, —S(O)—, —S(O)₂—, —N(H)—, —N(R)—, ═N—R—, —R—N═ and —C(X)— with R as defined previously, and with X representing an oxygen or sulfur atom or N—R, or combinations of said heteroatoms or groups, preferably chosen from: —O—C(O)—,
—C(O)—O—, —C(O)—N(H)—, —N(H)—C(O)—, —C(O)—N═, ═N—C(O)—, —N(H)—C(O)—N(H)—, —O—C(O)—N(H)—, —N(H)—C(O)—O—, —N(H)—C(NH)—NH—, and/or said alkyl radical being
optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) cationic or noncationic (hetero)cycle;
R₉ and R₁₀, which may be identical or different, represent:
a hydrogen atom,
a saturated or unsaturated, linear or branched C₁-C₆ alkyl radical, said alkyl radical:
optionally comprising one or more heteroatoms or groups, which may be identical or different, chosen from —O—, —S—, —S(O)—, —S(O)₂—, —N(H)—, —N(R)—, ═N—R—, —R—N═ and —C(X)— with R as defined previously, and with X representing an oxygen or sulfur atom or N—R, or combinations of said heteroatoms or groups, preferably chosen from: —O—C(O)—,
—C(O)—O—, —C(O)—N(H)—, —N(H)—C(O)—, —C(O)—N═, ═N—C(O)—, —N(H)—C(O)—N(H)—, —O—C(O)—N(H)—, —N(H)—C(O)—O—, —N(H)—C(NH)—NH—, and/or said alkyl radical being
optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) cationic or noncationic (hetero)cycle;
or R₉ and R₁₀ form, with the nitrogen atom that bears them, a 5- to 8-membered heterocycle, optionally substituted with one or more radicals, which may be identical or different, chosen from the following radicals: i) hydroxyl, ii) C₁-C₁₀ (hydroxy)alkyl, iii) C₁-C₆ (hydroxy)alkoxy, iv) amino
NH₂, v) C₁-C₄ mono and/or di(hydroxy)alkylamino, vi) thiol —SH, vii) C₁-C₄ alkylthiol, viii) carboxyl —COOH or carboxylate —COO⁻, M⁺ ix) C₁-C₄ alkylcarbonyl, x) sulfonic —SO₃H or sulfonate —SO₃⁻, M⁺, xi) amido —NH—C(O)CH₃, and at least one of the members of said heterocycle possibly representing a divalent oxo radical;
An⁻ denotes an anion or a mixture of anions for ensuring the electrical neutrality of the molecule, preferably chosen from halide ions such as bromide or chloride, methyl sulfate or toluenesulfonate ions, or a mixture of these ions;
M⁺ represents a cation or a mixture of cations for ensuring the electrical neutrality of the molecule, preferably chosen from sodium, potassium, calcium and ammonium.

2. The process as claimed claim 1, characterized in that the ammonium radical —N⁺RR′R″ according to the definition of the group A is chosen from trimethylammonium, triethylammonium, dimethylethylammonium, diethylmethylammonium, diisopropylmethylammonium, diethylpropylammonium, hydroxyethyldiethylammonium, β,β-dihydroxyethylmethylammonium and β,β,β-trihydroxyethylammonium radicals; preferably from trimethylammonium, triethylammonium, dimethylethylammonium, diethylmethylammonium, diisopropylmethyl ammonium and hydroxyethyldiethylammonium radicals.

3. The process as claimed in claim 1, characterized in that the cationic heterocycle according to the definition of the group A is chosen from imidazoliums, pyridiniums, piperaziniums, pyrrolidiniums, morpholiniums, pyrimidiniums, thiazoliums, benzimidazoliums, benzothiazoliums, oxazoliums, benzotriazoliums, pyrazoliums, triazoliums and benzoxazoliums; preferably from imidazoliums, pyridiniums, piperaziniums, pyrrolidiniums, morpholiniums, pyrimidiniums, thiazoliums and benzimidazoliums, and even more preferentially denotes an imidazolium or a piperazinium.

4. The process as claimed in claim 1, characterized in that the noncationic heterocycle according to the definition of the group A is chosen from imidazoles, pyridines, piperazines, pyrrolidines, morpholines, pyrimidines, thiazoles, benzimidazoles, benzothiazoles, oxazoles, benzotriazoles, pyrazoles, triazoles, benzoxazoles, piperidines, pyrroles and oxazolidinones; preferably from morpholines, piperidines, pyrrolidines, imidazoles, pyrroles, piperazines and 1,3-oxazolidin-2-ones.

5. The process as claimed in claim 1, characterized in that R₁, R₂, R₃, R₄, R₅, R₆, R₇ and R₈, which may be identical or different, represent:
a hydrogen atom,
a saturated or unsaturated, linear or branched C₁-C₆ alkyl radical, said alkyl radical being optionally substituted with one or more radicals, which may be identical or different, chosen from the following radicals: i) hydroxyl, ii) C₁-C₄ alkoxy, iii) amino —NH₂, iv) C₁-C₆ mono and/or di(hydroxy)alkylamino.

6. The process as claimed in claim 1, characterized in that R₁, R₂, R₃, R₄, R₅, R₆, R₇ and R₈ represent a hydrogen atom.

7. The process as claimed in claim 1, characterized in that R₉ and R₁₀, which may be identical or different, represent:
a hydrogen atom,
a saturated or unsaturated, linear or branched C₁-C₆ alkyl radical, said alkyl radical being optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino;
preferably, R₉ and R₁₀ are identical and represent a hydrogen atom.

8. The process as claimed in claim 1, characterized in that A represents a group —C(R₁₂)(R₁₃)—R₁₁ and R₁₁ forms with R₉, the nitrogen atom which bears R₉ and the carbon atoms which bear —N(R₉)—R₁₀ and R₁₁, a 5- to 8-membered cationic or noncationic heterocycle, said heterocycle being:
optionally substituted with one or more radicals, which may be identical or different, chosen from the following radicals: i) hydroxyl, ii) C₁-C₄ alkyl, iii) C₁-C₄ alkoxy and iv) an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl;
optionally at least one of the members of said heterocycle possibly representing a divalent oxo radical —C(O)—;
R₁₂ and R₁₃ being as defined in claim 1 and preferably denoting a hydrogen atom.

9. The process as claimed in claim 1, characterized in that the compound(s) of formula (I) are chosen from compounds (1) to (45), and also the geometrical or optical isomers thereof, the tautomers thereof, the organic or mineral acid or base salts thereof or the solvates thereof such as hydrates:

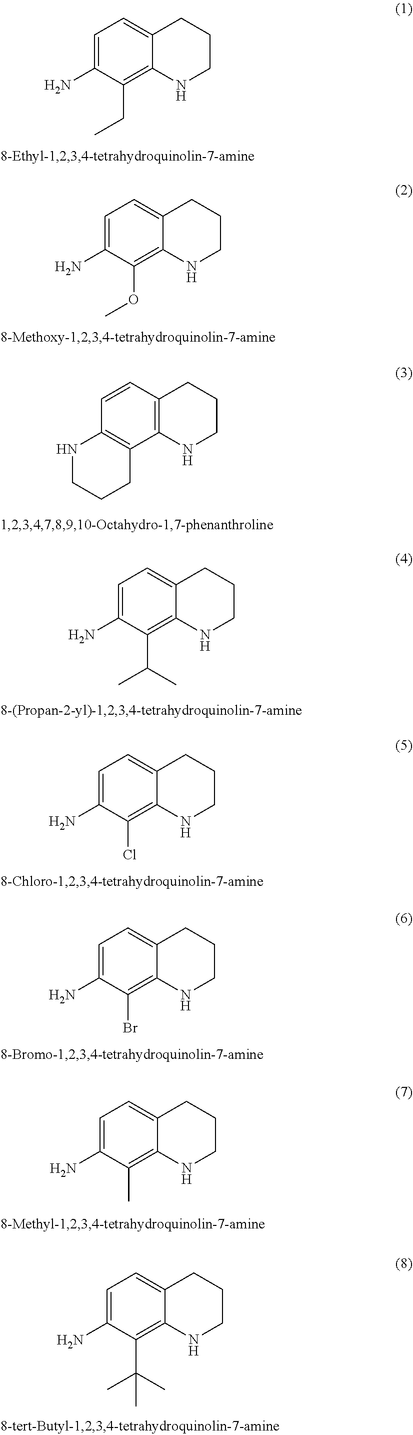

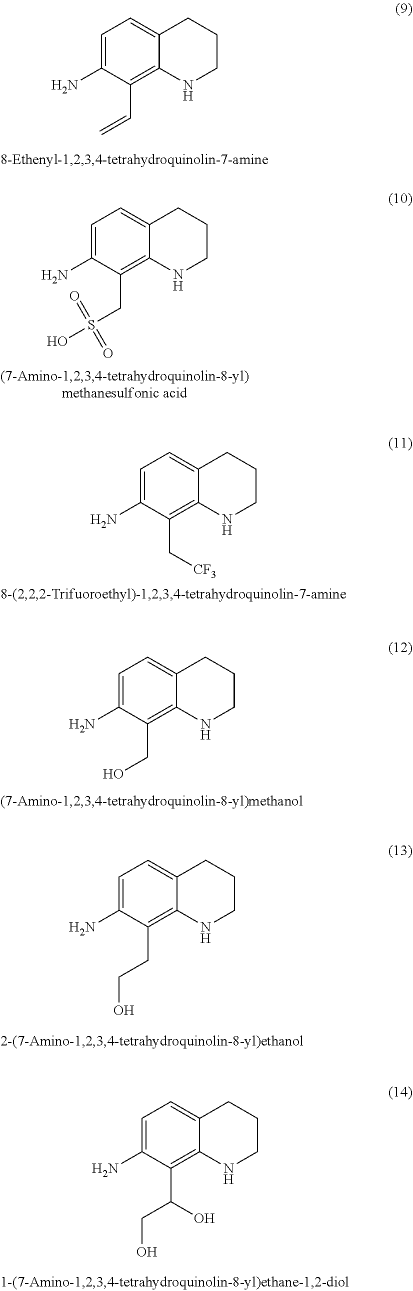 [SMILES: C=Cc1c(N)ccc2c1NCCC2 ; Nc1ccc2c(c1CS(=O)(=O)O)NCCC2 ; Nc1ccc2c(c1CC(F)(F)F)NCCC2 ; Nc1ccc2c(c1CO)NCCC2 ; Nc1ccc2c(c1CCO)NCCC2 ; Nc1ccc2c(c1C(O)CO)NCCC2]

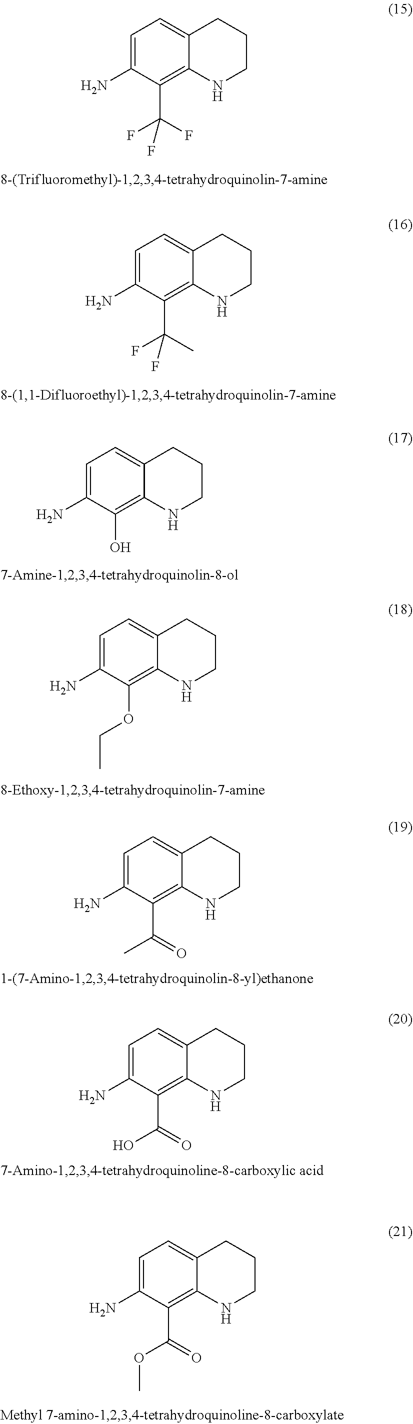

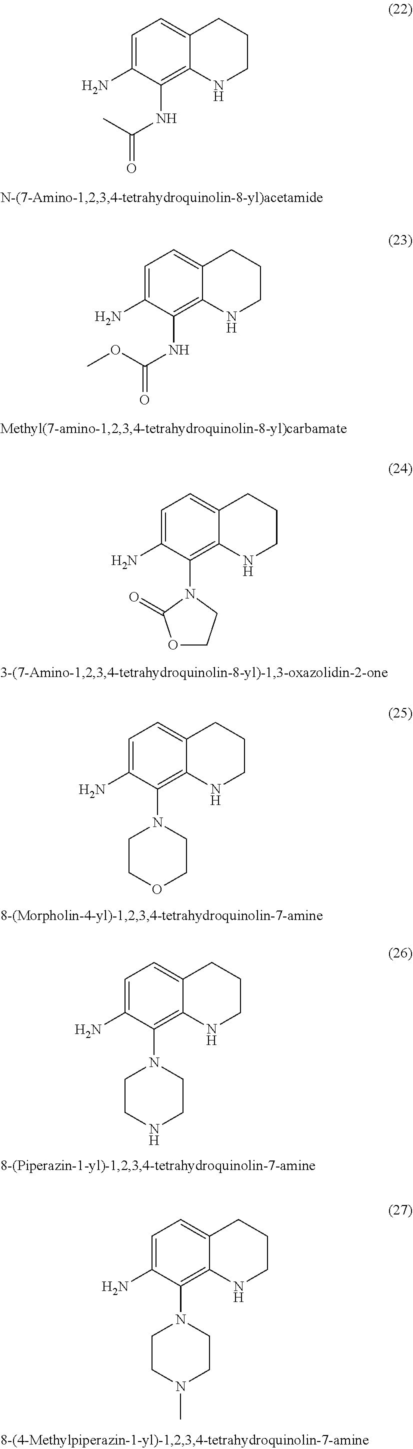 [SMILES: CC(=O)Nc1c(N)ccc2c1NCCC2 ; COC(=O)Nc1c(N)ccc2c1NCCC2 ; Nc1ccc2c(c1N1CCOC1=O)NCCC2 ; Nc1ccc2c(c1N1CCOCC1)NCCC2 ; Nc1ccc2c(c1N1CCNCC1)NCCC2 ; CN1CCN(c2c(N)ccc3c2NCCC3)CC1]

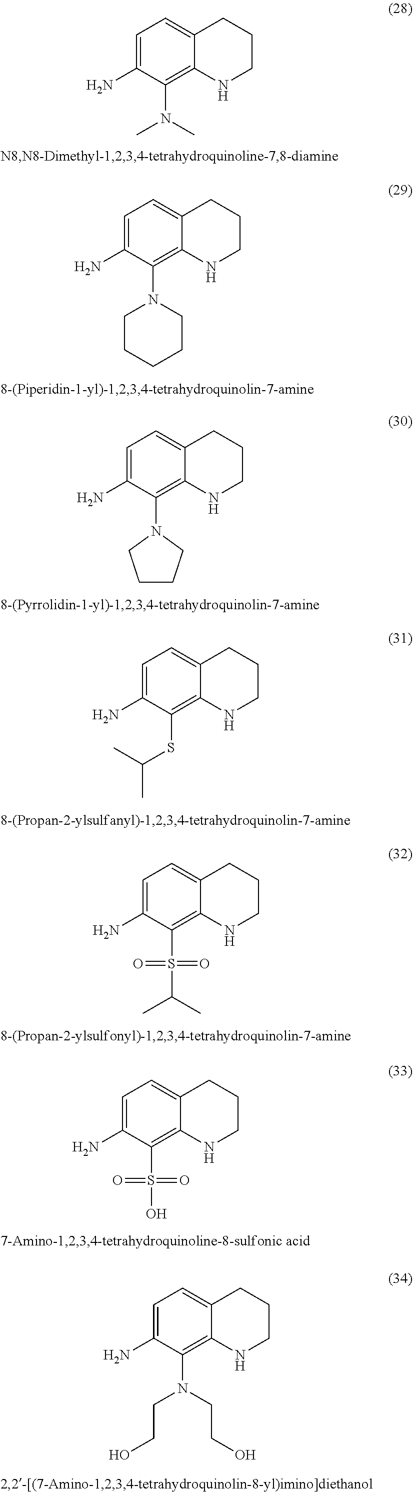

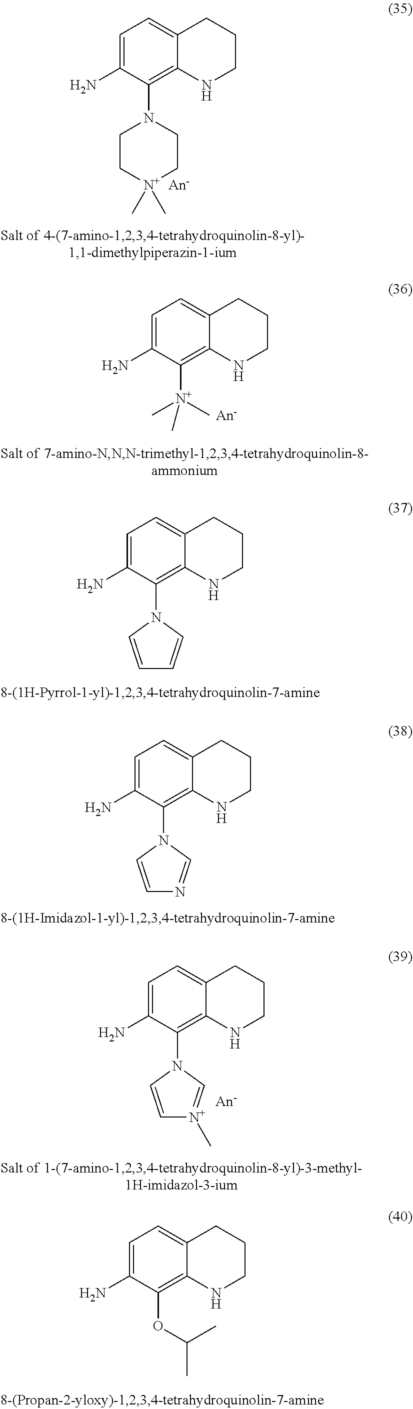 [SMILES: C[N+]1(C)CCN(c2c(N)ccc3c2NCCC3)CC1 ; C[N+](C)(C)c1c(N)ccc2c1NCCC2 ; Nc1ccc2c(c1-n1cccc1)NCCC2 ; Nc1ccc2c(c1-n1ccnc1)NCCC2 ; C[n+]1ccn(-c2c(N)ccc3c2NCCC3)c1 ; CC(C)Oc1c(N)ccc2c1NCCC2]

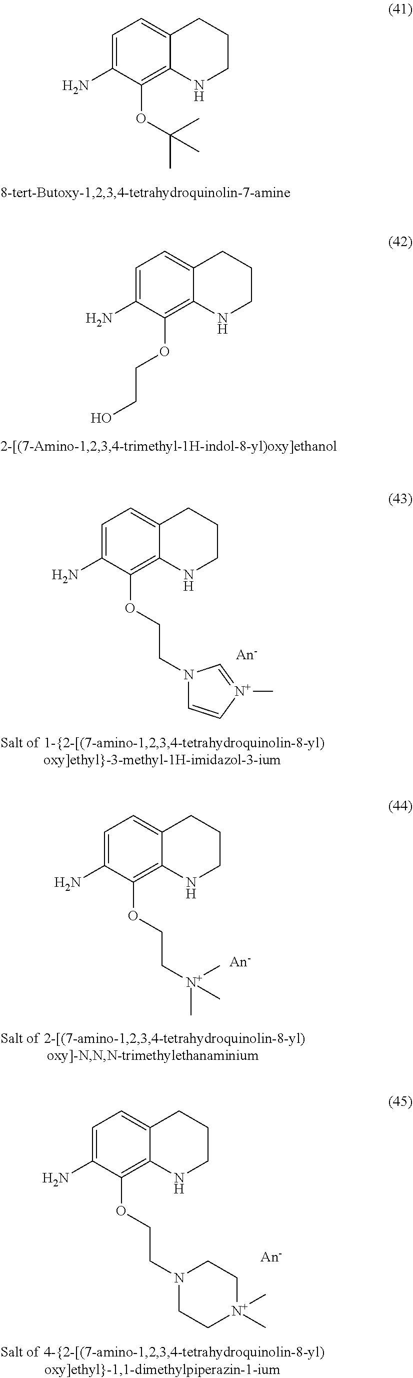 [SMILES: CC(C)(C)Oc1c(N)ccc2c1NCCC2 ; Nc1ccc2c(c1OCCO)NCCC2 ; C[n+]1ccn(CCOc2c(N)ccc3c2NCCC3)c1 ; C[N+](C)(C)CCOc1c(N)ccc2c1NCCC2 ; C[N+]1(C)CCN(CCOc2c(N)ccc3c2NCCC3)CC1]

10. The process as claimed in claim 1, comprising at least one step i) of applying to said keratin fibers one or more compounds of formula (I) and one or more oxidation bases.

11. The process as claimed in claim 1, comprising at least one step ii) of applying to said keratin fibers an oxidizing cosmetic composition comprising one or more chemical oxidizing agents, preferably chosen from hydrogen peroxide, urea peroxide, alkali metal bromates, persalts such as perborates and persulfates, peracids and oxidase enzymes (with the optional cofactors thereof), such as peroxidases, 2-electron oxidoreductases, for instance uricases, and 4-electron oxygenases, for instance laccases; more preferentially, the chemical oxidizing agent is hydrogen peroxide;
it being understood that between the step(s) i) and the step(s) ii), said fibers may be rinsed, and/or washed and then optionally dried.

12. The process as claimed in claim 1, comprising at least one step of simultaneously applying to said fibers:
one or more compounds of formula (I);
one or more chemical oxidizing agents;
optionally one or more oxidation bases.

13. A compound of formula (I), and also the organic or mineral acid or base salts thereof, the tautomeric forms thereof, the optical isomers thereof, the geometrical isomers thereof and/or the solvates thereof, as defined in claim 1, characterized in that:
A represents:
an iodine atom or a fluorine atom,
a carboxyl radical —COOH or carboxylate radical —COO⁻, M⁺,
a sulfonic radical —SO₃H or sulfonate radical —SO₃⁻M⁺, or alkylsulfonyl radical —S(O)₂—R with R representing a linear or branched (C₁-C₄)alkyl group,
a group —C(R₁₂)(R₁₃)—R₁₁ or —C(R₁₂)═C(R₁₁)—R₁₃, in which:
R₁₁, R₁₂ and R₁₃, which may be identical or different, represent:
a hydrogen atom, a halogen atom such as fluorine, chlorine, bromine or iodine, preferably fluorine,
a hydroxyl radical,
a linear or branched C₁-C₁₄ alkyl or alkenyl group, in particular C₁-C₈, preferably C₁-C₆, said alkyl or alkenyl group:
optionally comprising one or more heteroatoms or groups, which may be identical or different, chosen from —O—, —S—, —S(O)—,
—S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N═,
═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—NH—, and/or said alkyl or alkenyl group being
optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) fluoro —F, vi) sulfonic
SO₃H, or sulfonate —SO₃-M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl; ix) an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl;
it being understood that R₁₁, R₁₂ and R₁₃ cannot simultaneously denote a hydrogen atom,
or R₁₁ forms with R₉, the nitrogen atom which bears R₉ and the carbon atoms which bear —N(R₉)(R₁₀) and R₁₁, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical
(—C(O⁻), it being understood that if R₁₀, R₁₂ and R₁₃ denote a hydrogen atom, R₉ and R₁₁ cannot constitute a divalent radical —(CH₂)₃—;
a group —W′(R′₁₄) in which:
W′ represents an oxygen or sulfur atom,
R′₁₄ represents:
a hydrogen atom,
a linear or branched C₂-C₁₄ alkyl or alkenyl group, in particular C₂-C₈, preferably C₂-C₆, said alkyl or alkenyl group:
optionally interrupted with one or more heteroatoms or groups, which may be identical or different, chosen from —O—, —S—,
—S(O)—, —S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N═, ═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—N(H)—, and/or said alkyl or alkenyl group being
optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) fluoro —F, vi) sulfonic —SO₃H, or sulfonate —SO₃⁻M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An⁻, with R, R′ and R″, which may be identical or different, representing a (C₁-C₄)alkyl group optionally substituted with one or more hydroxyl groups, such as methyl, ethyl, propyl or 2-hydroxyethyl;
or R′₁₄ forms with R₉, the nitrogen atom which bears R₉ and the carbon atoms which bear —N(R₉)(R₁₀) and —W′—, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″,
An, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O⁻),
a group —N(R₁₅)—R′₁₅ in which R₁₅ and R′₁₅ independently represent:
a hydrogen atom,
a linear or branched C₁-C₁₄ alkyl or alkenyl group, in particular C₁-C₈, preferably C₁-C₆, said alkyl or alkenyl group:
optionally interrupted with one or more heteroatoms or groups, which may be identical or different, chosen from —O—, —S—,
—S(O)—, —S(O)₂—, —N(H)—, —N(R)—, ═N—R—, —R—N═ and —C(X)— with X representing an oxygen or sulfur atom or N—R and with R representing a (C₁-C₄)alkyl group optionally substituted with one or more hydroxyl groups, such as methyl, ethyl, propyl or 2-hydroxyethyl, or combinations of said heteroatoms or groups, preferably chosen from: —O—C(O)—, —C(O)—O—, —C(O)—N═, ═N—C(O)—, —C(O)—N(H)—, —N(H)—C(O)—, —N(H)—C(O)—N(H)—, —O—C(O)—N(H)—, —N(H)—C(O)—O—, —N(H)—C(NH)—NH—, and/or said alkyl or alkenyl group being
optionally substituted with one or more radicals, which may be identical or different, chosen from the following radicals: i) hydroxyl, ii) C₁-C₆ (hydroxy)alkoxy, iii) amino —NH₂, iv) C₁-C₆ mono and/or di(hydroxy)alkylamino, v) fluoro —F, vi) sulfonic
SO₃H, or sulfonate —SO₃⁻M⁺, vii) thiol —SH, viii) cationic or noncationic (hetero)cycle, said heterocycle being optionally substituted, in particular optionally substituted with one or more radicals, which may be identical or different, chosen from (hydroxy)(C₁-C₆)alkyl radicals such as methyl, ethyl, propyl or 2-hydroxyethyl, hydroxyl radicals, ammonium radicals —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl; ix) an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl;
it being understood that R₁₅ and R′₁₅ cannot simultaneously denote a hydrogen atom;
or R₁₅ forms with R₉, the nitrogen atom which bears R₉ and the carbon atoms which bear —N(R₉)(R₁₀) and —N(R′₁₅)—, a 5- to 8-membered cationic or noncationic heterocycle, said heterocycle being optionally substituted with one or more radicals, which may be identical or different, chosen from the following radicals: i) hydroxyl, ii) C₁-C₄ alkyl, iii) C₁-C₄ alkoxy, and iv) an ammonium radical —N⁺RR′R″,
An⁻, with R, R′ and R″, which may be identical or different, representing a (C₁-C₄)alkyl group optionally substituted with one or more hydroxyl groups, such as methyl, ethyl, propyl or 2-hydroxyethyl and at least one of the members of said heterocycle possibly representing a divalent oxo radical (—C(O⁻),
a carbonyl radical —C(O)—R₁₁, with R₁₁ as defined in claim 1,
an ester radical —C(O)O—R₁₁1, a thioester radical —C(O)S—R₁₁1 or a dithioester radical —C(S)S—R₁₁, with R₁₁ as defined in claim 1,
an optionally substituted cationic or noncationic (hetero)cycle,
an ammonium radical —N⁺RR′R″, An, with R, R′ and R″, which may be identical or different, representing a (C₁-C₄)alkyl group optionally substituted with one or more hydroxyl groups, such as methyl, ethyl, propyl or 2-hydroxyethyl;
with M⁺ as defined in claim 1,
it being understood that the compounds of formula (I) are other than:
7-amino-8-fluoro-1,2,3,4-tetrahydroquinoline 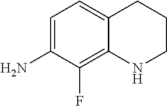 [SMILES: Nc1ccc2c(c1F)NCCC2]
7-amino-8-hydroxy-1,2,3,4-tetrahydroquinoline

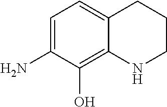 [SMILES: Nc1ccc2c(c1O)NCCC2]

14. The compound as claimed in claim 13, chosen from compounds (1), (4), (8), (9), (10), (11), (12), (13), (14), (15), (16), (18), (19), (20), (21), (22), (23), (24), (25), (26), (27), (28), (29), (30), (31), (32), (33), (34), (35), (36), (37), (38), (39), (40), (41), (42), (43), (44) and (45) and also the organic or mineral acid or base salts thereof, the tautomeric forms thereof, the optical isomers thereof, the geometrical isomers thereof and/or the solvates thereof such as the hydrates thereof.

15. A cosmetic composition comprising:
one or more compounds of formula (I) as defined in claim 1; and
one or more oxidation bases.

16. The composition as claimed in claim 15, characterized in that it comprises one or more chemical oxidizing agents, preferably chosen from hydrogen peroxide, urea peroxide, alkali metal bromates, persalts such as perborates and persulfates, peracids and oxidase enzymes (with the optional cofactors thereof), such as peroxidases, 2-electron oxidoreductases, for instance uricases, and 4-electron oxygenases, for instance laccases; more preferentially, the chemical oxidizing agent is hydrogen peroxide.

17. A multi-compartment device, or dyeing “kit”, comprising a first compartment containing one or more compounds of formula (I) as defined in claim 1, and a second compartment comprising one or more chemical oxidizing agents.
